(19) United States
(12) Patent Application Publication
(10) Pub. No.: US 2008/0229448 A1
(43) Pub. Date: Sep. 18, 2008

(54) Plant Stress Tolerance from Modified Ap2 Transcription Factors

(75) Inventors: Jeffrey M. Libby, Cupertino, CA (US); Oliver J. Ratcliffe, Oakland, CA (US); Neal I Gutterson, Oakland, CA (US); Robert A. Creelman, Castro Valley, CA (US)

Correspondence Address: MENDEL 2 C/O MOFO SF, 425 MARKET STREET, SAN FRANCISCO, CA, 94066, US

(73) Assignee: Mendel Biotechnology, Inc., Hayward, CA (US)

(21) Appl. No.: 11/793,466

(22) PCT Filed: Dec. 20, 2005
(86) PCT No.: PCT/US05/46492

§ 371 Date: Mar. 5, 2008

Related U.S. Application Data
(60) Provisional application No. 60/638,353, filed on Dec. 20, 2004.

(51) Int. Cl. C12N 15/82; A01H 5/00

(52) U.S. Cl. 800/279; 800/301; 800/289

(57) ABSTRACT

The invention relates to modified plant transcription factor polypeptides, polynucleotides that encode them, homologs from a variety of plant species, and methods of using the polynucleotides and polypeptides to produce transgenic plants having advantageous properties, including increased abiotic or biotic stress tolerance, as compared to wild-type or control plants. The modifications to the plant transcription factor sequences are responsible for producing fewer and less severe adverse morphological and developmental characteristics in plants overexpressing these sequences than would be caused by overexpressing the sequences without the modifications.

### FIELD OF THE INVENTION

The present invention relates to compositions and methods for producing plants with improved stress tolerance.

### BACKGROUND OF THE INVENTION

Abiotic stresses, including freezing temperatures, drought and high salinity, and biotic stresses, including disease, greatly limit the geographical locations where crops can be grown and cause significant losses in productivity on an annual basis. Many of the transcription factor genes that have been shown to confer abiotic or biotic stress tolerance in plants are AP2 family genes. However, overexpression of AP2 transcription factors tend to create plants that are smaller than wild-type. It is thus likely that there are conserved residues/motifs in all of these proteins that cause such effects. Ideally, AP2 polypeptides may be found or created that lack these conserved residues/motifs and can yet confer increased abiotic stress tolerance (e.g., freezing, chilling or drought tolerance) or biotic stress (disease tolerance) in plants with normal or near normal stature and fertility.

### SUMMARY OF THE INVENTION

The present invention relates to isolated polynucleotides that encode a mutated AP2 transcription factor polypeptide, including CBF and non-CBF AP2 transcription factors. In the description that follows, transgenic plants are described that are transformed with an expression vector comprising polynucleotides encoding AP2 transcription factors with particular mutations. These mutations may include, among others, point mutations, deletions, truncations, or fusions. Thus, a transgenic plant of the invention comprises and overexpresses an AP2 transcription factor that is mutated. These transgenic plants are generally larger than transgenic plants overexpressing the same AP2 transcription factor that has not been mutated (for example, in its native form). Although the AP2 transcription factors are mutated, they nonetheless have the ability to confer to the transgenic plant increased tolerance to an abiotic stress or greater resistance to a disease pathogen than a wild-type plant of the same species. However, unlike plants that overexpress non-mutated versions of the same transcription factor, the transgenic plants overexpressing the mutant forms of the protein are more similar in their morphology and size to wild-type plants grown for the same length of time. In some cases, the transgenic plants overexpressing the mutant form of the protein may be larger than the wild-type plant grown for the same length of time. Once produced, the transgenic plants overexpressing the mutated form of these proteins may be selected on the basis of their larger size than plants transformed with the non-mutated version of the protein, and their greater tolerance or resistance to an environmental stress than a wild-type plant of the same species.

### BRIEF DESCRIPTION OF THE SEQUENCE LISTING AND FIGURES

The Sequence Listing provides exemplary polynucleotide and polypeptide sequences of the invention. The traits associated with the use of the sequences are included in the Examples.

This application includes a Sequence Listing in paper form and in a computer text file named “MBI0074PCT.ST25.txt”. The file is 66 kilobytes in size, is included in CD-ROM1, Copy 1 and Copy 2, and was created and recorded on Dec. 20, 2005. The content of the Sequence Listing in the paper copy and in the computer file are identical, and the Sequence Listing is hereby incorporated by reference in its entirety.

FIG. 1 shows a conservative estimate of phylogenetic relationships among the orders of flowering plants (modified from Angiosperm Phylogeny Group (1998) Ann. Missouri Bot Gard. 84: 1-49). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales. FIG. 1 was adapted from Daly et al. (2001) Plant Physiol. 127: 1328-1333.

FIG. 2 shows a phylogenic dendogram depicting phylogenetic relationships of higher plant taxa, including clades containing tomato and Arabidopsis; adapted from Ku et al. (2000) Proc. Natl. Acad. Sci. 97: 9121-9126; and Chase et al. (1993) Ann. Missouri Bot. Gard. 80: 528-580.

FIG. 3A. T1 generation 35S::G47 (SEQ ID NO: 9) overexpressors (constitutive, direct promoter-fusion, construct P894, SEQ ID NO: 15) Lines 301, 305 and wild-type (WT) control at 31 days. The overexpressors were significantly smaller in size, later developing, and had upright, wide, curling leaves with vitreous inner rosette leaves relative to controls. Line 301 was shown to be significantly more salt and drought tolerant than controls.

FIG. 3B. T1 generation 35S::G47 (constitutive, site-directed mutation 2, construct P25733, SEQ ID NO: 38) lines 1104, 1105 and wild-type (WT) controls at 37 days after planting. The overexpressors were larger in stature with fuller rosettes, had wrinkled, curling leaves and were late in their development relative to controls. Line 1104 was shown to be more tolerant to desiccation than control plants.

FIG. 3C. T1 generation 35S::G47 (constitutive, site-directed mutation 4, construct P25735, SEQ ID NO: 39) lines 1062, 1063 and wild-type (WT) controls at 30 days after planting. The overexpressors had upright, curling leaves and were late in their development relative to the wild-type control. However, the plants overexpressing the G47 point mutations were of similar stature to the control plant and were more tolerant to cold during their germination than the controls.

FIG. 3D. T1 generation 35S::G47 (constitutive, site-directed mutation 4, construct P25735, SEQ ID NO: 39) lines 1606, 1609 and wild-type (WT) control plants 44 days after planting. The overexpressors had larger, fuller rosettes, twisted, curling leaves, and were later in their development relative to controls. Line 1609 was of larger stature at this stage of development. Line 1609 was also more tolerant to cold during germination, and to desiccation, than controls.

FIG. 4A. T1 generation 35S::G1792 (SEQ ID NO: 3) overexpressors (constitutive, direct promoter-fusion, construct P1695, SEQ ID NO: 12) Lines 305, 306 and wild-type (WT) control at 38 days. The overexpressors were significantly smaller in size and later developing, and had dark, twisting leaves and dark petioles. Line 305 was shown to be significantly more tolerant than controls to cold, desiccation, low nitrogen conditions, and to infection by Botrytis and Erysiphe.

FIG. 4B. T1 generation 35S::G1792 (constitutive, site-directed mutation 4, construct P25741, SEQ ID NO: 22) lines 1704, 1707 and wild-type (WT) control plants 31 days after planting. The overexpressors had large, grayer leaves with jagged margins, and were later developing relative to controls. Although it was of larger stature at this stage of development than control plants, line 1707 was more tolerant to cold and sucrose than controls.

FIG. 4C. T1 generation 35S::G1792 (constitutive, site-directed mutation 2, construct P25739, SEQ ID NO: 20) lines 987, 991 and wild-type (WT) control plants 28 days after planting. The overexpressors had dull, flat, serrated leaves relative to controls. Although it was at least as large as control plants at this stage of development, lines 987 and 991 were more tolerant to low nitrogen conditions than controls. Line 987 was also more tolerant to Erysiphe than controls, although not to the same extent as the many of the lines overexpressing the native G47 polypeptide.

FIG. 5A. T3 generation 35S::G28 (SEQ ID NO: 5) overexpressors (constitutive, direct promoter-fusion, construct P174, SEQ ID NO: 13) Line 55 wild-type (WT) control at 31 days. The overexpressors were smaller in size, later developing, and had darker green leaves relative to controls. Line 55 was shown to be more tolerant to Sclerotinia, Botrytis and Erysiphe than controls.

FIG. 5B. T1 generation 35S::G28 (constitutive, site-directed mutation 5, construct P25682, SEQ ID NO: 28) line 1085 and wild-type (WT) control plants 31 days after planting. The overexpressor had flat, pale leaves and developed early relative to controls. Although it was at least as large as control plants at this stage of development, line 1085 was shown to be more tolerant to Sclerotinia than controls.

FIG. 6A. T1 generation 35S::G867 ((SEQ ID NO: 7; two-component, constructs P6506, SEQ ID NO: 44 (35S::LexA-GAL4TA) and P7140, SEQ ID NO: 42 (opLexA::G867)) lines 1624, 1626 and wild type (WT) control are shown 24 days after planting. The overexpressors are small in size with upright leaves, but were shown to be more tolerant to salt, ABA and sucrose than controls.

FIG. 6B. T1 generation 35S::G867 (deletion variant, construct P21275, SEQ ID NO: 33) line 1038 and wild-type (WT) control plants 23 days after planting. The overexpressor had flat, pale rosette leaves that were larger than those of the controls. Although it was at least as large as control plants at this stage of development, line 1038 was shown to be more tolerant to cold than controls during seedling growth.

FIG. 7A. T1 generation 35S::G912 ((SEQ ID NO: 1; constitutive, two-component, constructs P6506, SEQ ID NO: 44, (35S::LexA-GAL4TA) and P3366, SEQ ID NO: 43 (opLexA::G912)) line 354 and wild type (WT) control are shown 32 days after planting. The overexpressor is tiny, dark and delayed in its development relative to the control.

FIG. 7B. T1 generation 35S::G912-GAL4 fused to the N-terminus of the protein (construct: P21197, SEQ ID NO: 18, an overexpression construct encoding a G912 clone that has a GAL4 transactivation domain fused at the N terminus). Lines 1341, 1351 and a wild-type (WT) controls are shown at 39 days after planting. The overexpressors are large, dark green, and late flowering. Lines 1341 and 1351 were more tolerant to drought, cold and freezing than the controls.

### DESCRIPTION OF THE INVENTION

The present invention relates to methods and compositions for producing transgenic plants with modified traits, particularly traits that address agricultural and food needs, by altering the expression of useful AP2 transcription factors. These transcription factors are modified by, for example, point mutations, truncations, deletions, or protein fusions, so that the transcription factors, when overexpressed, confer abiotic or biotic stress tolerance or resistance, respective, with minimal or no adverse morphological impact on the overexpressing plant. Another example of a modification is the replacement of transactivation motifs of a plant AP2 protein with alternate transactivation motifs that are sufficient to carry out the required transactivation functions of the transcription factor without interacting with other transcription factor components in an adverse manner. Transactivation motifs derived from other transcription factors, in some cases from non-plant sources, can be used for this purpose. The data presented herein represent the results obtained in experiments with modified transcription factor polynucleotides and polypeptides that may be expressed in plants for the purpose of reducing yield losses that arise from abiotic or biotic stress. A specific modification of AP2 polypeptides that confers abiotic or biotic stress tolerance with minimal or no adverse morphological impact on the overexpressmg plant includes the substitution of acidic amino acid residues with residues of lower acidity within a loop structure found within the AP2 domain (for example, the loop structure relative to positions 179-183 in G28, SEQ ID NO: 6).

In an important aspect, the present invention relates to polynucleotides and polypeptides, for example, for modifying phenotypes of plants, particularly those associated with increased stress tolerance. Throughout this disclosure, various information sources are referred to and/or are specifically incorporated. The information sources include scientific journal articles, patent documents, textbooks, and World Wide Web browser-active and inactive page addresses, for example. The reference to these information sources indicates that they can be used by one of skill in the art. The contents and teachings of each of the information sources can be relied on and used to make and use embodiments of the invention.

As used herein and in the appended Statements of the Inventions, the singular forms “a,” “an,” and “the” include plural reference unless the context clearly dictates otherwise. Thus, for example, a reference to “a plant” includes a plurality of such plants, and a reference to “a stress” is a reference to one or more stresses and equivalents thereof known to those skilled in the art, and so forth.

### DEFINITIONS

“Nucleic acid molecule” refers to an oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

A “polynucleotide” is a nucleic acid molecule comprising a plurality of polymerized nucleotides, e.g., at least about 15 consecutive polymerized nucleotides, and optionally at least about 30 consecutive nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single stranded.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as splicing and folding to obtain a functional protein or polypeptide. A gene may be isolated, partially isolated, or be found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and which may be used to determine the limits of the genetically active unit (Rieger et al. (1976) Glossary of Genetics and Cytogenetics: Classical and Molecular, 4th ed., Springer Verlag, Berlin). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic. “Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in or out of a cell than the polypeptide in its natural state in a wild-type cell (that is, not the result of a natural response of a wild-type plant), for example, more than about 5% or more enriched relative to wild type. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence. Additionally, the terms “homology” and “homologous sequence(s)” may refer to one or more polypeptide sequences that are modified by chemical or enzymatic means. The homologous sequence may be a sequence modified by lipids, sugars, peptides, organic or inorganic compounds, by the use of modified amino acids or the like. Protein modification techniques are illustrated in Ausubel et al. (eds.) Current Protocols in Molecular Biology, John Wiley & Sons (1998).

The terms “essentially homologous” or “sufficiently homologous” refer to polynucleotide or polypeptide sequences that are sufficiently duplicative of one another that the sequences produce the same or similar results when similarly expressed in plants. An example of a similar result is a comparable degree of a particular abiotic or biotic stress tolerance conferred when two sufficiently homologous sequences are expressed in two different plants. These sequences may include a sequence of the Sequence Listing of this application, or other comparatively similar sequences that confer similar functions in plants. Such sequences can also be used as a probe to isolate DNA's in other plants.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

With regard to polypeptides, the terms “substantial identity” or “substantially identical” may refer to sequences of sufficient similarity and structure to the transcription factors in the Sequence Listing to produce similar function when expressed, overexpressed, or knocked-out in a plant; in the present invention, this function is increased tolerance to conditions of limited light. Polypeptide sequences that are at least about 55% identical to the instant polypeptide sequences are considered to have “substantial identity” with the latter. Sequences having lesser degrees of identity but comparable biological activity are considered to be equivalents. The structure required to maintain proper functionality is related to the tertiary structure of the polypeptide. There are discreet domains and motifs within a transcription factor that must be present within the polypeptide to confer function and specificity. These specific structures are required so that interactive sequences will be properly oriented to retain the desired activity. “Substantial identity” may thus also be used with regard to subsequences, for example, motifs, that are of sufficient structure and similarity, being at least about 55% identical to similar motifs in other related sequences. Thus, AP2 family polypeptides have the physical characteristics of substantial identity along their full length and within their AP2 domains. These polypeptides also share functional characteristics, as the polypeptides within this clade bind to a transcription-regulating region of DNA and increase abiotic or biotic tolerance in a plant when the polypeptides are overexpressed.

“Alignment” refers to a number of nucleotide or amino acid residue sequences aligned by lengthwise comparison so that components in common (i.e., nucleotide bases or amino acid residues) may be visually and readily identified. The fraction or percentage of components in common is related to the homology or identity between the sequences. Alignments may be used to identify conserved domains and relatedness within these domains. An alignment may suitably be determined by means of computer programs known in the art, such as MacVector (1999) (Accelrys, Inc., San Diego, Calif.).

A “conserved domain” or “conserved region” as used herein refers to a region in heterologous polynucleotide or polypeptide sequences where there is a relatively high degree of sequence identity between the distinct sequences. AP2 domains are examples of conserved domains. With respect to polynucleotides encoding presently disclosed transcription factors, a conserved domain is preferably at least 10 base pairs (bp) in length.

A “conserved domain”, with respect to presently disclosed polypeptides refers to a domain within a transcription factor family that exhibits a higher degree of sequence homology, such as at least 55% sequence similarity, and more preferably at least 60% sequence identity, and even more preferably at least 62%, or at least about 56%, or at least about 59%, or at least about 65%, or at least about 70%, or at least about 77%, or at least about 78%, or at least about 80%, or at least about 81%, or at least about 82%, or at least about 83%, or at least about 84%, or at least about 86%, or at least about 88%, or at least about 89%, or at least about 90%, or at least about 93%, or at least about 95% amino acid residue sequence identity of a polypeptide of consecutive amino acid residues. A fragment or domain can be referred to as outside a conserved domain, outside a consensus sequence, or outside a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

Conserved domains such as conserved DNA binding domains may be used to identify closely-related sequences that have a particular sequence identity with the similar domain in a particular AP2 transcription factor, that is, domains with a degree of relatedness that may be used as indicators of similar or identical function. Hurley et al. (Hurley et al. (2001) Trends Biochem. Sci. 27: 48-53) noted that “structure determination led to the hypothesis that the TULP core domain was the DNA-binding domain of a transcription factor, which was bome out by functional assays” (Hurley, supra). As evidence that Hurley et al. recognized that conserved domains are structural features associated with evolutionary-relatedness and function, the authors stated that “[b]ioinformatics-based discoveries of new signaling domain families sometimes define biochemical function in a clear-cut way, as when one or more family members correspond to protein fragments whose activity has been previously characterized” (Hurley, supra). Hurley et al. also summed up the state-of-the-art in the understanding that conserved domains are effective indicators and predictors of related protein function: “[o]nce the function of a particular domain from one protein is well understood, powerful and testable inferences can be made as to the function of the many other proteins that contain that domain” (Hurley, supra, which describes “hypothesis-driven experiments” for determining related functions of signaling and DNA-binding domains).

Thus, as one of ordinary skill in the art recognizes, conserved domains may be identified as regions or domains of identity to a specific consensus sequence, and by using alignment methods well known in the art, the conserved domains of plant transcription factors may be determined.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of the hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y. (“Sambrook”); and by Haymes et al., “Nucleic Acid Hybridization: A Practical Approach”, IRL Press, Washington, D.C. (1985).

In general, stringency is determined by the temperature, ionic strength, and concentration of denaturing agents (e.g., formamide) used in a hybridization and washing procedure (for a more detailed description of establishing and determining stringency, see below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, transcription factors having 60% identity, or more preferably greater than about 70% identity, most preferably 72% or greater identity with disclosed transcription factors.

Regarding the terms “paralog” and “ortholog”, homologous polynucleotide sequences and homologous polypeptide sequences may be paralogs or orthologs of the polynucleotide or polypeptide sequences of the invention. Orthologs and paralogs are evolutionarily related genes that have similar sequence and similar functions. Orthologs are structurally related genes in different species that are derived by a speciation event. Paralogs are structurally related genes within a single species that are derived by a duplication event. Sequences that are sufficiently similar to one another will be appreciated by those of skill in the art and may be based upon percentage identity of the complete sequences, percentage identity of a conserved domain or sequence within the complete sequence, percentage similarity to the complete sequence, percentage similarity to a conserved domain or sequence within the complete sequence, and/or an arrangement of contiguous nucleotides or peptides particular to a conserved domain or complete sequence. Sequences that are sufficiently similar to one another will also bind in a similar manner to the same DNA binding sites of transcriptional regulatory elements using methods well known to those of skill in the art.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “www.tigr.org” or “http://www.tigr.org/TIGRFAMs/Explanations.shtml” under the heading “Terms associated with TIGRFAMs”.

The term “variant”, as used herein, may refer to polynucleotides or polypeptides that differ from the presently disclosed polynucleotides or polypeptides, respectively, in sequence from each other, and as set forth below.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a transcription factor nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor. Thus, it will be readily appreciated by those of skill in the art, that any of a variety of polynucleotide sequences is capable of encoding the transcription factors and transcription factor homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, for example, replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (U.S. Pat. No. 5,840,544).

“Modulates” refers to a change in activity (biological, chemical, or immunological) or lifespan resulting from specific binding between a molecule and either a nucleic acid molecule or a protein.

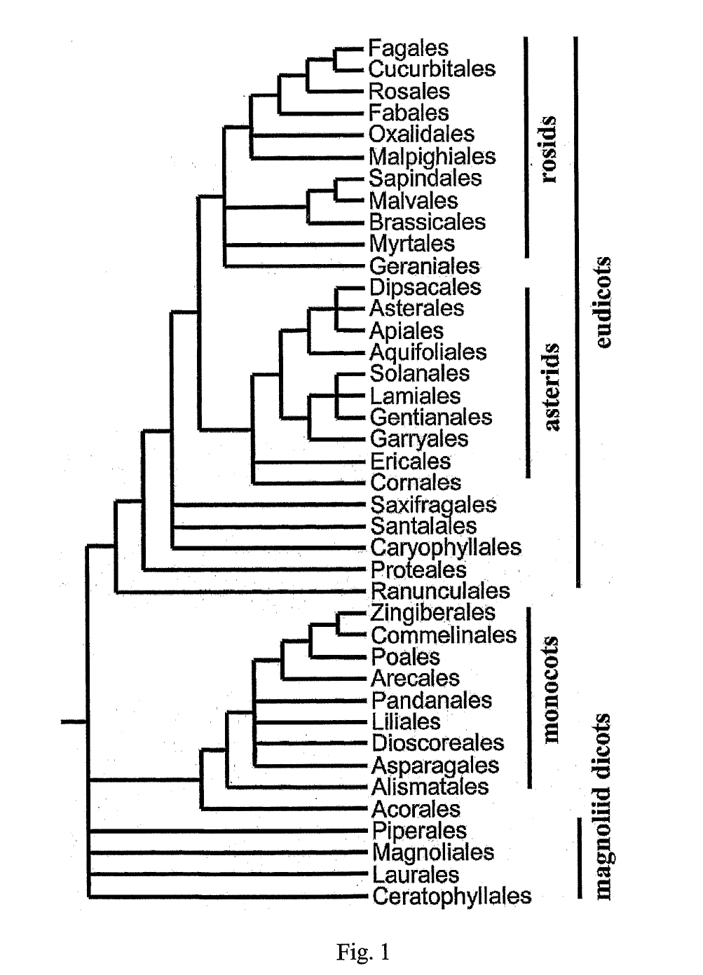

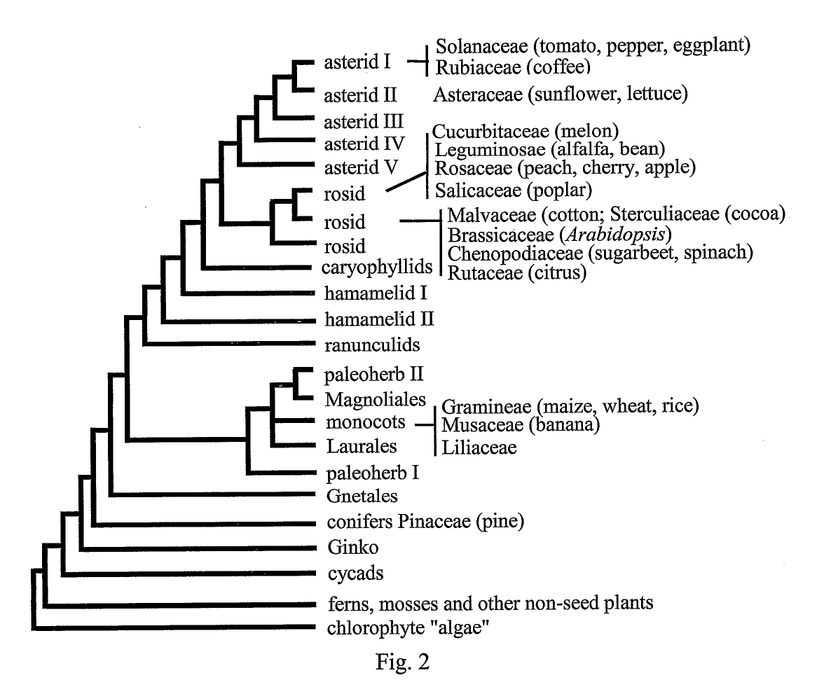

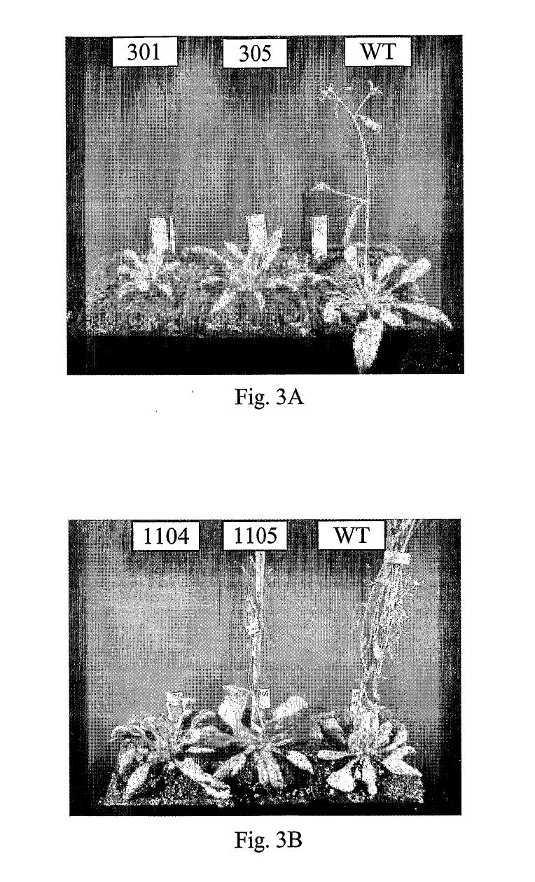

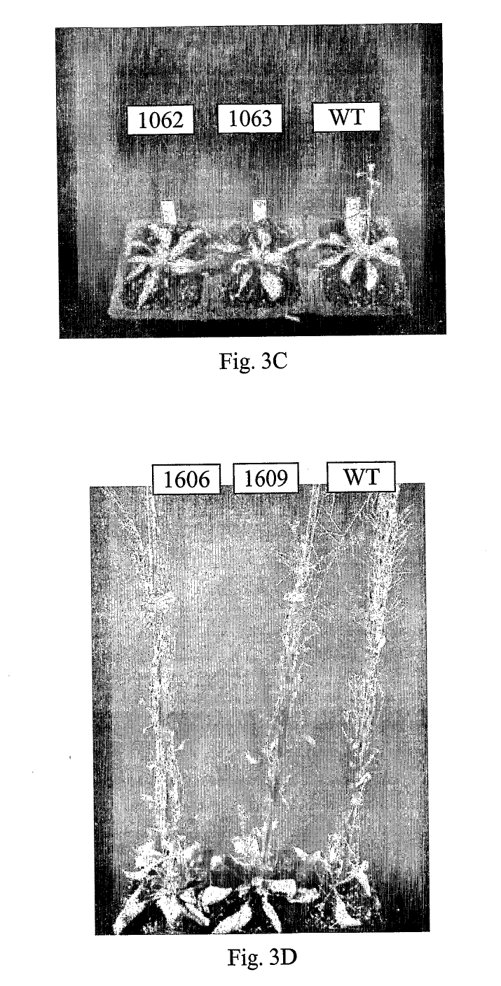

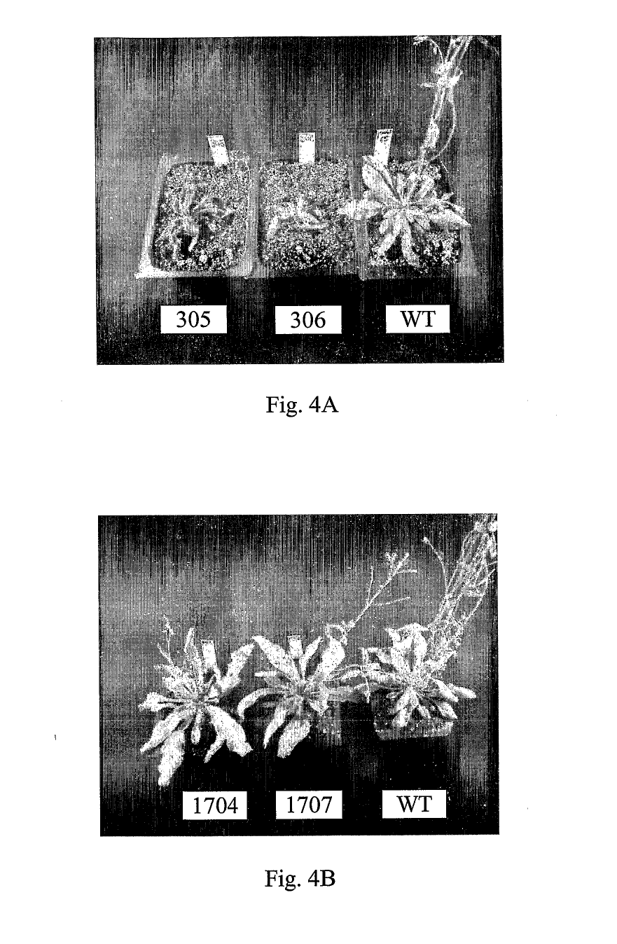

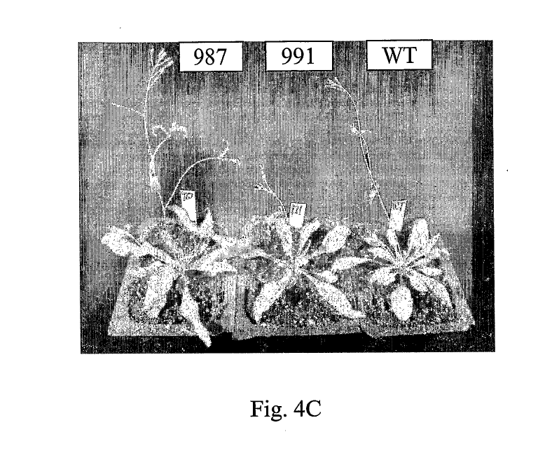

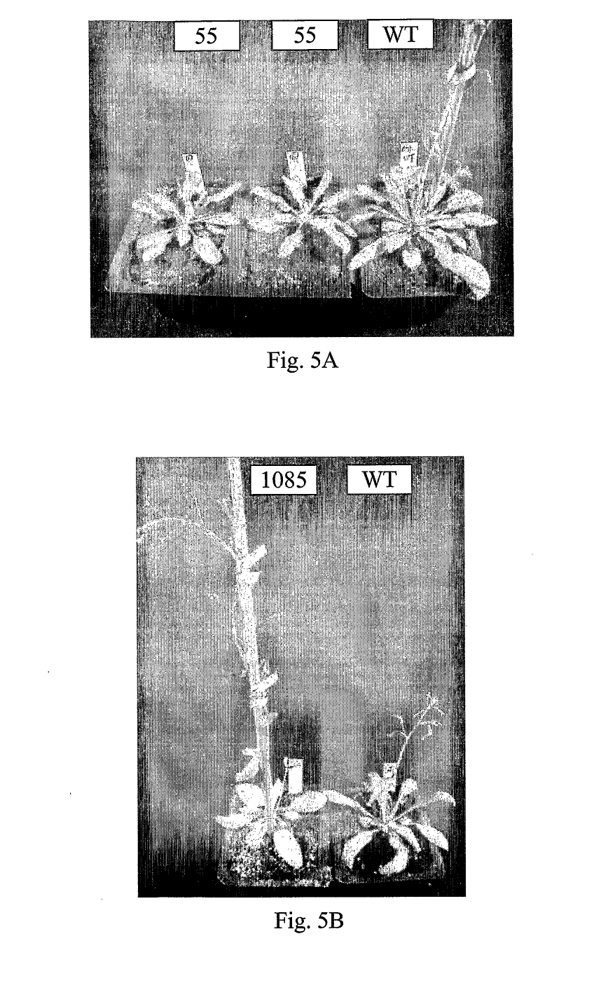

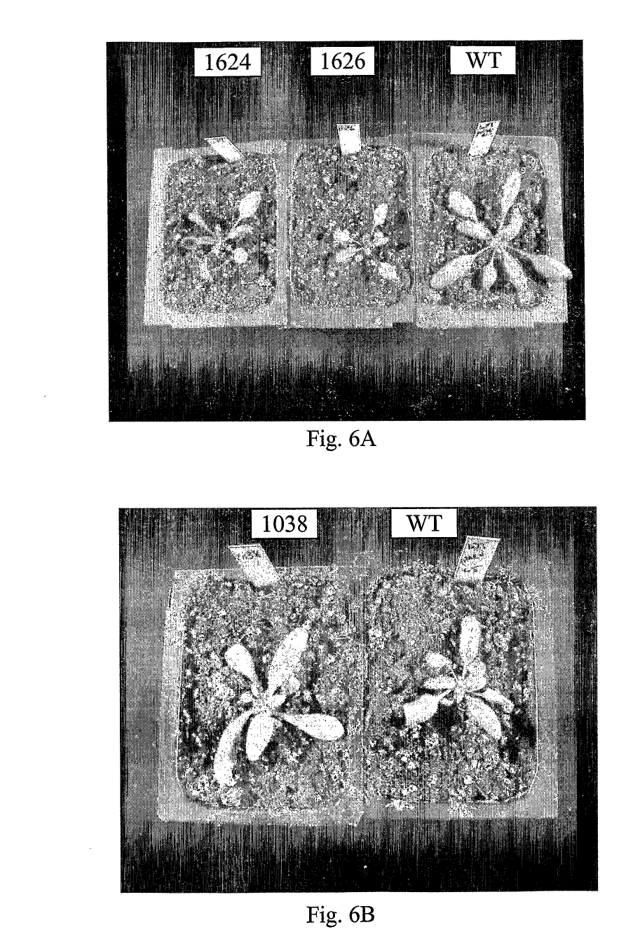

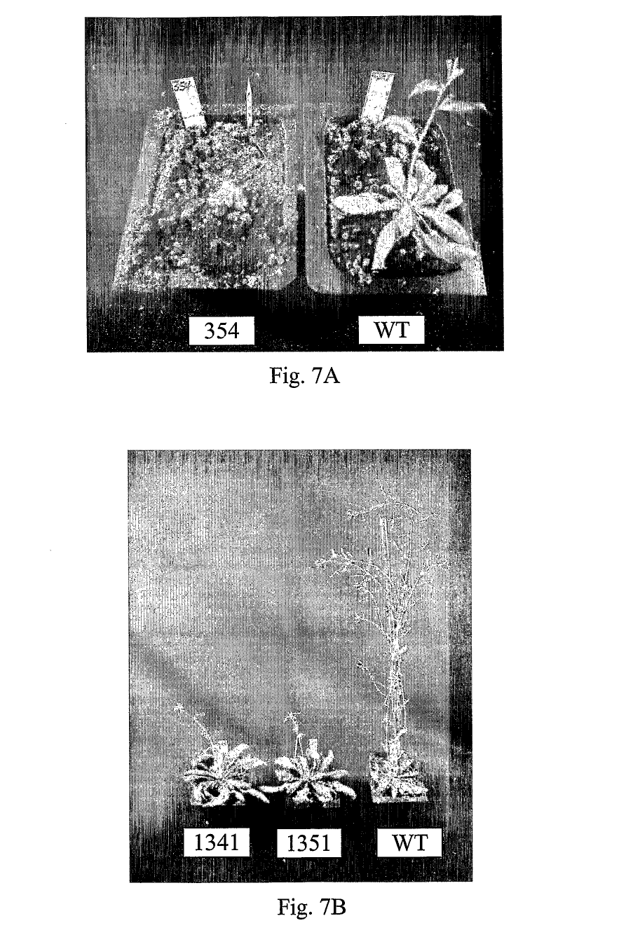

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat), progeny plants derived from seed, fruit (the mature ovary), plant tissue (for example, vascular tissue, ground tissue, and the like) and cells (for example, guard cells, egg cells, and the like), and progeny derived from tissue or cells. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (for example, as in FIG. 1, adapted from Daly et al. (2001) Plant Physiol. 127: 1328-1333; FIG. 2, adapted from Ku et al. (2000) Proc. Natl. Acad. Sci. 97: 9121-9126; and also as in Tudge in The Variety of Life, Oxford University Press, New York, N.Y. (2000) pp. 547-606).

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the expression of the polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, for example, a plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a transcription factor expression is altered, e.g., in that it has been knocked out, overexpressed, or ectopically expressed. In the present invention, transgenic plants are generally compared to controls such as wild-type plants of the same species that grown for the same length of time and grown under the same conditions as the transgenic plant.

A “control plant” as used in the present invention refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, preferably at least about 30 nucleotides, more preferably at least about 50 nucleotides, of any of the sequences provided herein.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length. Exemplary polypeptide fragments are the first twenty consecutive amino acids of a mammalian protein encoded by are the first twenty consecutive amino acids of the transcription factor polypeptides listed in the Sequence Listing. Exemplary polynucleotide fragments are the first sixty consecutive nucleotides of the transcription factor polynucleotides listed in the Sequence Listing. Exemplary fragments also include fragments that comprise a region that encodes a conserved domain of a transcription factor, for example, an AP2 domain such as found at amino acid residues 144-208 of G28, SEQ ID NO: 6.

The invention also encompasses production of DNA sequences that encode transcription factors and transcription factor derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding transcription factors or any fragment thereof.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, for example, by employing Northern analysis, RT-PCR, microarray gene expression assays, or reporter gene expression systems, or by agricultural observations such as limited light conditions or other abiotic stress tolerance or yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, or at least about a 100%, or an even greater difference compared with a wild-type plant. It is known that there can be a natural variation in the modified trait. Therefore, the trait modification observed entails a change of the normal distribution of the trait in the plants compared with the distribution observed in wild-type plants.

When two or more plants are “morphologically similar” or “similar in morphology”, they have comparable forms or appearances, including analogous features such as dimension, height, width, mass, root mass, shape, glossiness, color, stem diameter, leaf size, leaf dimension, leaf density, internode distance, branching, root branching, number and form of inflorescences, and other macroscopic characteristics at a particular stage of growth. If the plants are morphologically similar at all stages of growth, they are also “developmentally similar”. It may be difficult to distinguish two plants that are genotypically distinct but morphologically similar based on morphological characteristics alone.

Plants of “similar size” or “similar stature” or that are “similar in size” are also difficult to distinguish based on observations of height, volume or biomass alone.

The term “transcript profile” refers to the expression levels of a set of genes in a cell in a particular state, particularly by comparison with the expression levels of that same set of genes in a cell of the same type in a reference state. For example, the transcript profile of a particular transcription factor in a suspension cell is the expression levels of a set of genes in a cell repressing or overexpressing that transcription factor compared with the expression levels of that same set of genes in a suspension cell that has normal levels of that transcription factor. The transcript profile can be presented as a list of those genes whose expression level is significantly different between the two treatments, and the difference ratios. Differences and similarities between expression levels may also be evaluated and calculated using statistical and clustering methods.

“Ectopic expression” or “altered expression” in reference to a polynucleotide indicates that the pattern of expression in, for example, a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the terms “ectopic expression” or “altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more transcription factors are under the control of a strong expression signal, such as one of the promoters described herein (for example, the cauliflower mosaic virus 35S transcription initiation region). Overexpression may occur throughout a plant or in specific tissues of the plant, depending on the promoter used, as described below.

Overexpression may take place in plant cells normally lacking expression of polypeptides functionally equivalent or identical to the present transcription factors. Overexpression may also occur in plant cells where endogenous expression of the present transcription factors or functionally equivalent molecules normally occurs, but such normal expression is at a lower level. Overexpression thus results in a greater than normal production, or “overproduction” of the transcription factor in the plant, cell or tissue.

The term “transcription-regulating region” refers to a DNA regulatory sequence that regulates expression of one or more genes in a plant when a transcription factor having one or more specific binding domains binds to the DNA regulatory sequence. Transcription factors of the present invention possess for example, an AP2 domain that comprises a transcription-regulating region. The transcription factors of the invention also comprise an amino acid subsequence that forms a transcription activation domain that regulates expression of abiotic or biotic stress tolerance genes in a plant when the transcription factor binds to the regulating region.

The term “cold stress” refers to a decrease in ambient temperature, including a decrease to freezing temperatures, which causes a plant to attempt to acclimate itself to the decreased ambient temperature.

The term “dehydration stress” refers to drought, desiccation, freezing, high salinity and other conditions that cause a decrease in cellular water potential in a plant.

### DETAILED DESCRIPTION

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding site or DNA-binding site motif (for example, in Riechmann et al. (2000) Science 290: 2105-2110). The plant transcription factors may belong to, for example, the AP2 or other transcription factor families.

Generally, the transcription factors encoded by the present sequences are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to abiotic or biotic stress tolerance. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from any source whether natural, synthetic, semi-synthetic or recombinant. The sequences of the invention may also include fragments of the present amino acid sequences that function in a manner similar to the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, for example, mutation reactions, PCR reactions, or the like; as substrates for cloning for example, including digestion or ligation reactions; and for identifying exogenous or endogenous modulators of the transcription factors. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, for example, genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) Genes Development 11: 3194-3205, and Peng et al. (1999) Nature, 400: 256-261. In addition, many others have demonstrated that an Arabidopsis transcription factor expressed in an exogenous plant species elicits the same or very similar phenotypic response (for example, in Fu et al. (2001) Plant Cell 13: 1791-1802; Nandi et al. (2000) Curr. Biol. 10: 215-218; Coupland (1995) Nature 377: 482-483; and Weigel and Nilsson (1995) Nature 377: 482-500).

In another example, Mandel et al. (1992) Cell 71-133-143, and Suzuki et al. (2001) Plant J. 28: 409-418, teach that a transcription factor expressed in another plant species elicits the same or very similar phenotypic response of the endogenous sequence, as often predicted in earlier studies of Arabidopsis transcription factors in Arabidopsis (Mandel et al. (1992) supra; Suzuki et al. (2001) supra).

Other examples include Miller et al. ((2001) Plant J. 28: 169-179)), Kim et al. ((2001) Plant J. 25: 247-259), Kyozuka and Shimamoto ((2002) Plant Cell Physiol. 43: 130-135), Boss and Thomas ((2002) Nature, 416: 847-850)), He et al. ((2000) Transgenic Res. 9: 223-227)), and Robson et al. ((2001) Plant J. 28: 619-631).

In yet another example, Gilmour et al. (1998) Plant J. 16: 433-442, teach an Arabidopsis AP2 transcription factor, CBF1, which, when overexpressed in transgenic plants, increases plant freezing tolerance. Jaglo et al. (2001) Plant Physiol. 127: 910-917, further identified sequences in Brassica napus that encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues, P-K-K/R-P/R-A-G-R-x-K-F-x-E-T-R-H-P and D-S-A-W-R, which bracket the AP2/EREBP DNA binding domains of the proteins and distinguish them from other members of the AP2 protein family (Jaglo et al. (2001) supra) (that is, non-CBF AP2 polypeptide sequences encoded by non-CBF AP2 polynucleotides).

Transcription factors mediate cellular responses and control traits through altered expression of genes containing cis-acting nucleotide sequences that are targets of the introduced transcription factor. It is well appreciated in the art that the effect of a transcription factor on cellular responses or a cellular trait is determined by the particular genes whose expression is either directly or indirectly (for example, by a cascade of transcription factor binding events and transcriptional changes) altered by transcription factor binding. In a global transcription analysis comparing a standard condition with one in which a transcription factor is overexpressed, the resulting transcript profile associated with transcription factor overexpression is related to the trait or cellular process controlled by that transcription factor. For example, the PAP2 and other genes in the MYB family have been shown to control anthocyanin biosynthesis through regulation of the expression of genes known to be involved in the anthocyanin biosynthetic pathway (Bruce et al. (2000) Plant Cell, 12: 65-79; Borevitz et al. (2000) Plant Cell 12: 2383-93). Further, global transcript profiles have been used successfully as diagnostic tools for specific cellular states (for example, cancerous vs. non-cancerous; Bhattacharjee et al. (2001) Proc. Natl. Acad. Sci., USA 98: 13790-13795; Xu et al. (2001) Proc. Natl. Acad. Sci., USA, 98: 15089-15094). Consequently, it is evident to one skilled in the art that similarity of transcript profile upon overexpression of different transcription factors would indicate similarity of transcription factor function.

### Polypeptides and Polynucleotides of the Invention

The present invention relates to polynucleotides and polypeptides that may be used to increase a tolerance or resistance to environmental stress in a plant that is morphologically and developmentally similar to a control plant. The present invention provides, among other things, transcription factors (TFs), transcription factor homolog polypeptides, isolated or recombinant polynucleotides encoding the polypeptides, and/or novel sequence variant polypeptides or polynucleotides encoding novel variants of transcription factors derived from the specific sequences of the invention.

### AP2 Domain Transcription Factors

The AP2 family is a large transcription factor gene family includes 145 transcription factors (Weigel (1995) Plant Cell 7: 388-389; Okamuro et al. (1997) Proc. Natl. Acad. Sci. USA 94: 7076-7081; Riechmann and Meyerowitz (1998) Biol. Chem. 379:633-646; Riechmann et al. (2000) Science 290: 2105-2110). This family of proteins affects the regulation of a wide range of morphological and physiological processes, including the acquisition of stress tolerance. The AP2 family can be divided into three subfamilies:
(a) The APETALA2 subfamily is related to the APETALA2 protein itself (Jofuko et al. (1994) Plant Cell 6: 1211-1225), characterized by the presence of two AP2 DNA binding domains, and contains 14 genes.
(b) The AP2/ERF is the largest subfamily and includes 125 genes, many of which are involved in abiotic (DREB subgroup) and biotic (ERF subgroup) stress responses (Ohme-Takagi and Shinshi (1995) Plant Cell 7: 173-182; Zhou et al. (1995) Cell 83: 925-935; Stockinger et al. (1997) Proc. Natl. Acad. Sci. USA 94: 1035-1040; Jaglo-Ottosen et al. (1998) Science 280: 104-106; Finkelstein et al. (1998) Plant Cell 10: 1043-1054).
(c) The RAV subfamily, which contains six genes that have a B3 DNA binding domain in addition to the AP2 DNA binding domain.

The AP2 polynucleotides of the invention may be ectopically expressed in plant cells, and the changes in the expression levels of a number of genes, polynucleotides, and/or proteins of the plant cells observed. Therefore, the polynucleotides and polypeptides can be employed to change expression levels of genes, polynucleotides, and/or proteins of plants. These polypeptides and polynucleotides may be employed to modify a plant's characteristics, particularly abiotic or biotic stress tolerance.

The present invention thus relates to DNA, including isolated DNA, that encodes mutant or variant polypeptides capable of binding to a DNA regulatory sequence that regulates expression of one or more environmental stress tolerance genes in a plant. The mutant or variant polypeptides confer abiotic or biotic stress tolerance to a plant when overexpressed, but the plant retains morphological and developmental similarity to a control or wild-type plant of the same species that does not overexpress an AP2 family polypeptide. This may not the case with plants that overexpress wild-type, non-variant or non-mutated AP2 polynucleotides or polypeptides; the latter plants may have a number of defects including low fertility or seed production, or altered size.

The isolated DNA sequence may exist in a variety of forms, including in a plasmid or vector. The plasmid or vector can include a promoter that regulates expression of the regulatory gene. In one variation of this embodiment, the DNA regulatory sequence encodes an AP2 family transcription factor having a characteristic AP2 domain. AP2 sequences appear to be conserved in plants with some degree of variability from plant to plant. In one variation of this embodiment, the gene sequence of the invention encodes a mutant or variant AP2 polypeptide. In other variations of this invention, the gene sequence encodes a truncated variant of an AP2 polypeptide, or an AP2 gene sequence encoding a GAL4-AP2 polypeptide fusion product. In each of these cases, various embodiments of the invention have been shown to confer abiotic or biotic stress tolerance with little or no adverse impact on plant morphology.

Promoters can be used to overexpress the mutant or variant AP2 polypeptide, change the environmental conditions under which the mutant or variant AP2 polypeptide is expressed, or enable the expression of the mutant or variant AP2 polypeptide to be induced, for example by the addition of an exogenous inducing agent. Promoters can also be used to cause the mutant or variant AP2 polypeptide to be expressed at selected times during a plant's life. Tissue-specific promoters can be used to cause the mutant or variant AP2 polypeptide to be expressed in selected tissues. For example, flower-, fruit- and seed-specific promoters can be used to cause the mutant or variant AP2 polypeptide to be selectively expressed in flowers, fruits or seeds of the plant.

The present invention also relates to methods for using the DNA and mutant or variant AP2 polypeptides to regulate expression of one or more native or non-native environmental stress tolerance genes in a plant. These methods may include introducing DNA encoding a mutant or variant AP2 polypeptide capable of binding to a DNA regulatory sequence into a plant, introducing a promoter into a plant that regulates expression of the AP2 polypeptide, introducing a DNA regulatory sequence into a plant to which a mutant or variant AP2 polypeptide can bind, and/or introducing one or more environmental stress tolerance genes into a plant whose expression is regulated by a DNA regulatory sequence.

The present invention also relates to recombinant cells, plants and plant materials (e.g., plant tissue, seeds) into which one or more gene sequences encoding a mutant or variant AP2 polypeptide have been introduced, as well as cells, plants and plant materials within which recombinant AP2 polypeptides encoded by these gene sequences are expressed. By introducing a gene sequence encoding a mutant or variant AP2 polypeptide into a plant, a mutant or variant AP2 polypeptide can be overexpressed or ectopically expressed within the plant. The mutant or variant AP2 polypeptide is capable of regulating expression of one or more stress tolerance genes in the plant, which is morphologically and developmentally similar to a control or wild-type plant. Regulation of expression can include causing one or more stress tolerance genes to be expressed under different conditions compared to the expression of those genes in the plant's native state, increasing a level of expression of one or more stress tolerance genes, and/or causing the expression of one or more stress tolerance genes to be inducible by an exogenous agent or environmental condition.

The present invention relates to the mutant or variant AP2 polypeptides encoded by the DNA. The DNA and mutant or variant AP2 polypeptides may be naturally occurring (that is, a naturally occurring mutation has taken place within a plant), or artificially mutagenized or varied (for the latter, a number of possible methods may be used such as by creating truncations or fusions). One embodiment of the invention relates to a mutant or variant AP2 polypeptide capable of selectively binding to a DNA regulatory sequence that regulates expression of one or more environmental stress tolerance genes in a plant, preferably by selectively binding to a DNA regulatory sequence that regulates the environmental stress tolerance genes. Because of the nature of the mutation or variation, this plant retains morphological and developmental similarity to a control or wild-type plant of the same species. In one variation, the mutant or variant AP2 polypeptide is a non-naturally occurring polypeptide formed by combining an amino acid sequence capable of binding to a regulatory sequence, with an amino acid sequence that forms a transcription activation region that regulates expression of one or more environmental stress tolerance genes.

In one variation, the stress tolerance regulatory gene sequence encodes a polypeptide homolog of a mutant or variant AP2 polypeptide disclosed herein. Preferably, the subsequence encoding the AP2 is preferably a homolog of a subsequence encoding one of the mutant or variant AP2 polypeptides disclosed herein.

In another variation, the DNA sequence encoding the mutant or variant AP2 polypeptide comprises an AP2 domain that is sufficiently homologous to G28, SEQ ID NO: 6, that the mutant or variant AP2 polypeptide is capable of binding to DNA regulatory sequence. A plant overexpressing this variant polypeptide is generally more morphologically similar to a wild-type or control plant than a transgenic plant overexpressing the G28 polypeptide.

The mutant or variant AP2 polypeptide may be derived from any plant that possesses a genome encoding an AP2 polypeptide that confers increased abiotic or biotic stress tolerance when the AP2 polypeptide is overexpressed. The Examples provided below demonstrate that different variations may confer abiotic or biotic stress tolerance in plants of wild-type morphology (or nearly wild-type morphology) and fertility. These variations include, for example, point mutations (including deletions, additions and substitutions), truncations, and GAL4-polypeptide fusions. Other variations in the amino acid sequence of the AP2 polypeptide may also confer abiotic or biotic stress tolerance in plants of wild-type morphology (or nearly wild-type morphology) and fertility, and are encompassed by the invention.

### Producing Polypeptides

The polynucleotides of the invention include sequences that encode transcription factors and transcription factor homolog polypeptides and sequences complementary thereto, as well as unique fragments of coding sequence, or sequence complementary thereto. Such polynucleotides can be, for example, DNA or RNA, the latter including mRNA, cRNA, synthetic RNA, genomic DNA, cDNA synthetic DNA, oligonucleotides, etc. The polynucleotides are either double-stranded or single-stranded, and include either, or both sense (i.e., coding) sequences and antisense (i.e., non-coding, complementary) sequences. The polynucleotides include the coding sequence of a transcription factor, or transcription factor homolog polypeptide, in isolation, in combination with additional coding sequences (e.g., a purification tag, a localization signal, as a fusion-protein, as a pre-protein, or the like), in combination with non-coding sequences (for example, introns or inteins, regulatory elements such as promoters, enhancers, terminators, and the like), and/or in a vector or host environment in which the polynucleotide encoding a transcription factor or transcription factor homolog polypeptide is an endogenous or exogenous gene.

A variety of methods exist for producing the polynucleotides of the invention. Procedures for identifying and isolating DNA clones are well known to those of skill in the art, and are described in, for example, Berger and Kimmel, Guide to Molecular Cloning Techniques, Methods in Enzymology, vol. 152 Academic Press, Inc., San Diego, Calif. (“Berger and Kimmel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, Ausubel et al. eds., Current Protocols, a joint venture between Greene Publishing Associates, Inc. and John Wiley & Sons, Inc., (supplemented through 2000) (“Ausubel”).

Alternatively, polynucleotides of the invention can be produced by a variety of in vitro amplification methods adapted to the present invention by appropriate selection of specific or degenerate primers. Examples of protocols sufficient to direct persons of skill through in vitro amplification methods, including the polymerase chain reaction (PCR), the ligase chain reaction (LCR), Qβ-replicase amplification and other RNA polymerase mediated techniques (for example, NASBA). Protocols for the production of the homologous nucleic acids of the invention are found in Berger and Kimmel (supra), Sambrook (supra), and Ausubel (supra), as well as Mullis et al. (1987) PCR Protocols A Guide to Methods and Applications (Innis et al. eds) Academic Press Inc. San Diego, Calif. (1990) (Innis). Improved methods for cloning in vitro amplified nucleic acids are described in Wallace et al. U.S. Pat. No. 5,426,039. Improved methods for amplifying large nucleic acids by PCR are summarized in Cheng et al. (1994) Nature 369: 684-685, and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase (e.g., in Ausubel, Sambrook and Berger, all supra).

Alternatively, polynucleotides and oligonucleotides of the invention can be assembled from fragments produced by solid-phase synthesis methods. Typically, fragments of up to approximately 100 bases are individually synthesized and then enzymatically or chemically ligated to produce a desired sequence, e.g., a polynucleotide encoding all or part of a transcription factor. For example, chemical synthesis using the phosphoramidite method is described, e.g., by Beaucage et al. (1981) Tetrahedron Letters 22: 1859-1869; and Matthes et al. (1984) EMBO J. 3: 801-805. According to such methods, oligonucleotides are synthesized, purified, annealed to their complementary strand, ligated and then optionally cloned into suitable vectors. And if so desired, the polynucleotides and polypeptides of the invention can be custom ordered from any of a number of commercial suppliers.

### Homologous Sequences

Sequences homologous, i.e., that share significant sequence identity or similarity, to those provided in the Sequence Listing, derived from Arabidopsis thaliana or from other plants of choice, are also an aspect of the invention. Homologous sequences can be derived from any plant including monocots and dicots and in particular agriculturally important plant species, including but not limited to: crops such as soybean, wheat, corn (maize), potato, cotton, rice, rape, oilseed rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits and fruit trees (such as apple, peach, pear, cherry and plum) and brassicas (such as broccoli, cabbage, cauliflower, Brussels sprouts, and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassaya, turnip, radish, yam, and sweet potato; and beans. The homologous sequences may also be derived from woody species, such pine, poplar and eucalyptus, or mint or other labiates. In addition, homologous sequences may be derived from plants that are evolutionarily related to crop plants, but which may not have yet been used as crop plants. Examples include deadly nightshade (Atropa belladona), related to tomato; jimson weed (Datura strommium), related to peyote; and teosinte (Zea species), related to corn (maize).

### Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360). For example, a clade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) supra). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (also, for example, in Mount (2001), in Bioinformatics: Sequence and Genome Analysis Cold Spring Harbor Laboratory Press, Cold Spring Harbor, N.Y. page 543).

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species.

It is well known in the art that protein function can be classified using phylogenetic analysis of gene trees combined with the corresponding species. Functional predictions can be greatly improved by focusing on how the genes became similar in sequence (i.e., evolution) rather than on the sequence similarity itself (Eisen, (1998) Genome Res. 8: 163-167): “[t]he first step in making functional predictions is the generation of a phylogenetic tree representing the evolutionary history of the gene of interest and its homologs. Such trees are distinct from clusters and other means of characterizing sequence similarity because they are inferred by techniques that help convert patterns of similarity into evolutionary relationships . . . . After the gene tree is inferred, biologically determined functions of the various homologs are overlaid onto the tree. Finally, the structure of the tree and the relative phylogenetic positions of genes of different functions are used to trace the history of functional changes, which is then used to predict functions of [as yet] uncharacterized genes” (Eisen, supra).

Thus, once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Transcription factor gene sequences are conserved across diverse eukaryotic species lines (Goodrich et al. (1993) Cell 75: 519-530; Lin et al. (1991) Nature 353: 569-571; Sadowski et al. (1988) Nature 335: 563-564). Plants are no exception to this observation; diverse plant species possess transcription factors that have similar sequences and functions.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence). An example of such highly related AP2 paralogs is the CBF family, with four well-defined members in Arabidopsis, CBF1, CBF2, CBF3 (Gilmour et al. (1998) supra; Jaglo et al. (1998) Plant Physiol. 127: 910-917), and G912 (CBF4; SEQ ID NO: 2; GenBank accession number BAB 11047) and at least one ortholog in Brassica napus, all of which control pathways involved in both freezing and drought stress (Gilmour et al. (1998) supra; Jaglo et al. (1998) Plant Physiol. 127: 910-917).

The following references represent a small sampling of the many studies that demonstrate that conserved transcription factor genes from diverse species are likely to function similarly (i.e., regulate similar target sequences and control the same traits), and that transcription factors may be transformed into diverse species to confer or improve traits.

(1) The Arabidopsis NPR1 gene regulates systemic acquired resistance (SAR) (Cao et al. (1997) Cell 88: 57-63); over-expression of NPR1 leads to enhanced resistance in Arabidopsis. When either Arabidopsis NPR1 or the rice NPR1 ortholog was overexpressed in rice (which, as a monocot, is diverse from Arabidopsis), challenge with the rice bacterial blight pathogen Xanthomonas oryzae pv. Oryzae, the transgenic plants displayed enhanced resistance (Chem et al. (2001) Plant J. 27: 101-113). NPR1 acts through activation of expression of transcription factor genes, such as TGA2 (Fan and Dong (2002) Plant Cell 14: 1377-1389).

(2) E2F genes are involved in transcription of plant genes for proliferating cell nuclear antigen (PCNA). Plant E2Fs share a high degree of similarity in amino acid sequence between monocots and dicots, and are even similar to the conserved domains of the animal E2Fs. Such conservation indicates a functional similarity between plant and animal E2Fs. E2F transcription factors that regulate meristem development act through common cis-elements, and regulate related (PCNA) genes (Kosugi and Ohashi, (2002) Plant J. 29: 45-59).

(3) The ABI5 gene (ABA insensitive 5) encodes a basic leucine zipper factor required for ABA response in the seed and vegetative tissues. Co-transformation experiments with ABI5 cDNA constructs in rice protoplasts resulted in specific transactivation of the ABA-inducible wheat, Arabidopsis, bean, and barley promoters. These results demonstrate that sequentially similar ABI5 transcription factors are key targets of a conserved ABA signaling pathway in diverse plants. (Gampala et al. (2001) J. Biol. Chem. 277: 1689-1694).

(4) Sequences of three Arabidopsis GAMYB-like genes were obtained on the basis of sequence similarity to GAMYB genes from barley, rice, and L. temulentum. These three Arabidopsis genes were determined to encode transcription factors (AtMYB33, AtMYB65, and AtMYB101) and could substitute for a barley GAMYB and control α-amylase expression (Gocal et al. (2001) Plant Physiol. 127: 1682-1693).

(5) The floral control gene LEAFY from Arabidopsis can dramatically accelerate flowering in numerous dictoyledonous plants. Constitutive expression of Arabidopsis LEAFY also caused early flowering in transgenic rice (a monocot) with a heading date that was 26-34 days earlier than that of wild-type plants. These observations indicate that floral regulatory genes from Arabidopsis are useful tools for heading date improvement in cereal crops (He et al. (2000) Transgenic Res. 9: 223-227).

(6) Bioactive gibberellins (GAs) are essential endogenous regulators of plant growth. GA signaling tends to be conserved across the plant kingdom. GA signaling is mediated via GAI, a nuclear member of the GRAS family of plant transcription factors. Arabidopsis GAI has been shown to function in rice to inhibit gibberellin response pathways (Fu et al. (2001) Plant Cell 13: 1791-1802).

(7) The Arabidopsis gene SUPERMAN (SUP), encodes a putative transcription factor that maintains the boundary between stamens and carpels. By over-expressing Arabidopsis SUP in rice, the effect of the gene's presence on whorl boundaries was shown to be conserved. This demonstrated that SUP is a conserved regulator of floral whorl boundaries and affects cell proliferation (Nandi et al. (2000) Curr. Biol. 10: 215-218).

(8) Maize, petunia and Arabidopsis myb transcription factors that regulate flavonoid biosynthesis are very genetically similar and affect the same trait in their native species, therefore sequence and function of these myb transcription factors correlate with each other in these diverse species (Borevitz et al. (2000) Plant Cell 12: 2383-2394).

(9) Wheat reduced height-1 (Rht-B1/Rht-D1) and maize dwarf-8 (d8) genes are orthologs of the Arabidopsis gibberellin insensitive (GAI) gene. Both of these genes have been used to produce dwarf grain varieties that have improved grain yield. These genes encode proteins that resemble nuclear transcription factors and contain an SH2-like domain, indicating that phosphotyrosine may participate in gibberellin signaling. Transgenic rice plants containing a mutant GAI allele from Arabidopsis have been shown to produce reduced responses to gibberellin and are dwarfed, indicating that mutant GAI orthologs could be used to increase yield in a wide range of crop species (Peng et al. (1999) Nature 400: 256-261).

(10) Distinct Arabidopsis transcription factors, including the AP2 sequences G1792 (SEQ ID NO: 4; US Patent Application 20040098764) and G867 (SEQ ID NO: 8; US Patent Application 20040098764), as well as the CAAT-family G482 polypeptide (US Patent Application 20040045049), and the AT-hook polypeptide G1073 (U.S. Pat. No. 6,717,034), have been shown to confer biotic (e.g., G1792) or abiotic (e.g., G482, G867, G1073, G1792) stress tolerance when the sequences are overexpressed. The polypeptides sequences belong to distinct clades of transcription factor polypeptides that include members from diverse species. In each case, a significant number of orthologous sequences derived from both dicots and monocots have been shown to confer tolerance to various abiotic stresses when the sequences were overexpressed (unpublished data, and as noted in these applications).

Transcription factors that are homologous to the listed sequences will typically share at least 55% sequence similarity, and more preferably at least 60% sequence identity, or at least 62%, or at least about 56%, or at least about 59%, or at least about 65%, or at least about 70%, or at least about 77%, or at least about 78%, or at least about 80%, or at least about 81%, or at least about 82%, or at least about 83%, or at least about 84%, or at least about 86%, or at least about 88%, or at least about 89%, or at least about 90%, or at least about 93%, or at least about 95% amino acid residue sequence identity of a polypeptide of consecutive amino acid residues with the listed sequences, or with the listed sequences but excluding or outside a known consensus sequence or consensus DNA-binding site, or with the listed sequences excluding one or all conserved domains.

At the nucleotide level, the sequences will typically share at least about 40% nucleotide sequence identity, preferably at least about 50%, about 60%, about 70% or about 80% sequence identity, and more preferably about 85%, about 90%, about 95% or about 97% or more sequence identity to one or more of the listed sequences, or to a listed sequence but excluding or outside a known consensus sequence or consensus DNA-binding site, or outside one or all conserved domain. The degeneracy of the genetic code enables major variations in the nucleotide sequence of a polynucleotide while maintaining the amino acid sequence of the encoded protein. AP2 domains of closely-related sequences within the AP2 transcription factor family may exhibit a higher degree of sequence homology. Transcription factors that are homologous to the listed sequences should share at least 30%, or at least about 60%, or at least about 75%, or at least about 80%, or at least about 90%, or at least about 95% amino acid sequence identity over the entire length of the polypeptide or the homolog.

Percent identity can be determined electronically, e.g., by using the MEGALIGN program (DNASTAR, Inc. Madison, Wis.). The MEGALIGN program can create alignments between two or more sequences according to different methods, for example, the clustal method (for example, in Higgins and Sharp (1988) Gene 73: 237-244). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (e.g., U.S. Pat. No. 6,262,333).

Other techniques for alignment are described in Methods Enzymol., vol. 266, “Computer Methods for Macromolecular Sequence Analysis” (1996), ed. Doolittle, Academic Press, Inc., San Diego, Calif., USA. Preferably, an alignment program that permits gaps in the sequence is utilized to align the sequences. The Smith-Waterman is a type of algorithm that permits gaps in sequence alignments (e.g., Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (e.g., Hein (1990) Methods Enzymol. 183: 626-645). Identity between sequences can also be determined by other methods known in the art, e.g., by varying hybridization conditions (e.g., US Patent Application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against BLOCKS (Bairoch et al. (1997) Nucleic Acids Res. 25: 217-221), PFAM, and other databases that contain previously identified and annotated motifs, sequences and gene functions. Methods that search for primary sequence patterns with secondary structure gap penalties (Smith et al. (1992) Protein Engineering 5: 35-51) as well as algorithms such as Basic Local Alignment Search Tool (BLAST; Altschul (1993) J. Mol. Evol. 36: 290-300; Altschul et al. (1990) J. Mol. Biol. 215: 403-410), BLOCKS (Henikoff and Henikoff (1991) Nucleic Acids Res. 19: 6565-6572), Hidden Markov Models (HMM; Eddy (1996) Curr. Opin. Str. Biol. 6: 361-365; Sonnhammer et al. (1997) Proteins 28: 405-420), and the like, can be used to manipulate and analyze polynucleotide and polypeptide sequences encoded by polynucleotides. These databases, algorithms and other methods are well known in the art and are described in Ausubel et al. (1997; Short Protocols in Molecular Biology, John Wiley & Sons, New York, N.Y., unit 7.7) and in Meyers (1995; Molecular Biology and Biotechnology, Wiley VCH, New York, N.Y., p 856-853).

Another method for identifying or confirming that specific homologous sequences control the same function is by comparison of the transcript profile(s) obtained upon overexpression or knockout of two or more related transcription factors. Since transcript profiles are diagnostic for specific cellular states, one skilled in the art will appreciate that genes that have a highly similar transcript profile (e.g., with greater than 50% regulated transcripts in common, more preferably with greater than 70% regulated transcripts in common, most preferably with greater than 90% regulated transcripts in common) will have highly similar functions. Fowler et al. (2002, Plant Cell, 14: 1675-79) have shown that three paralogous AP2 family genes (CBF1, CBF2 and CBF3), each of which is induced upon cold treatment, and each of which can condition improved freezing tolerance, have highly similar transcript profiles. Once a transcription factor has been shown to provide a specific function, its transcript profile becomes a diagnostic tool to determine whether putative paralogs or orthologs have the same function. Furthermore, methods using manual alignment of sequences similar or homologous to one or more polynucleotide sequences or one or more polypeptides encoded by the polynucleotide sequences may be used to identify regions of similarity and AP2 domains. Such manual methods are well-known of those of skill in the art and can include, for example, comparisons of tertiary structure between a polypeptide sequence encoded by a polynucleotide, which comprises a known function, with a polypeptide sequence encoded by a polynucleotide sequence, which has a function not yet determined. Such examples of tertiary structure may comprise predicted α-helices, β-sheets, amphipathic helices, leucine zipper motifs, zinc finger motifs, proline-rich regions, cysteine repeat motifs, and the like.

Orthologs and paralogs of presently disclosed transcription factors may be cloned using compositions provided by the present invention according to methods well known in the art. cDNAs can be cloned using mRNA from a plant cell or tissue that expresses one of the present transcription factors. Appropriate mRNA sources may be identified by interrogating Northern blots with probes designed from the present transcription factor sequences, after which a library is prepared from the mRNA obtained from a positive cell or tissue. Transcription factor-encoding cDNA is then isolated using, for example, PCR, using primers designed from a presently disclosed transcription factor gene sequence, or by probing with a partial or complete cDNA or with one or more sets of degenerate probes based on the disclosed sequences. The cDNA library may be used to transform plant cells. Expression of the cDNAs of interest is detected using, for example, methods disclosed herein such as microarrays, Northern blots, quantitative PCR, or any other technique for monitoring changes in expression. Genomic clones may be isolated using similar techniques to those.

### Identifying Polynucleotides or Nucleic Acids by Hybridization

Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited above.

Encompassed by the invention are polynucleotide sequences that are capable of hybridizing to the polynucleotide sequences of the invention, including any of the transcription factor polynucleotides within the Sequence Listing, and fragments thereof under various conditions of stringency (e.g., in Wahl and Berger (1987) Methods Enzymol. 152: 399-407; and Kimmel (1987) Methods Enzymol. 152: 507-511). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art (for example, in Sambrook et al. (1989) “Molecular Cloning: A Laboratory Manual” (2nd ed., Cold Spring Harbor Laboratory); Berger and Kimmel, eds., (1987) “Guide to Molecular Cloning Techniques”, in Methods Enzymol. 152: 467-469; and Anderson and Young (1985) “Quantitative Filter Hybridisation” in: Hames and Higgins, ed., Nucleic Acid Hybridisation, A Practical Approach. Oxford, IRL Press, 73-111).

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

Tₘ(° C.)=81.5+16.6(log [Na+])+0.41(% G+C)−0.62(% formamide)−500/L (I) DNA-DNA

Tₘ(° C.)=79.8+18.5(log [Na+])+0.58(% G+C)+0.12(% G+C)²−0.5(% formamide)−820/L (II) DNA-RNA

Tₘ(° C.)=79.8+18.5(log [Na+])+0.58(% G+C)+0.12(% G+C)²−0.35(% formamide)−820/L (III) RNA-RNA

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at (Tₘ₋5°)C. to (Tₘ₋20°)C., moderate stringency at (Tₘ−20°)C. to (Tₘ-35)° C. and low stringency at (Tₘ₋35°)C. to (Tₘ₋50°)C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at (Tₘ-25°)C. for DNA-DNA duplex and (Tₘ-15°)C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 min, or about 0.1×SSC, 0.1% SDS at 65° C. and washing twice for 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over about 30 min. Even higher stringency wash conditions are obtained at 65° C. to 68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (for example, US Patent Application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Estimates of homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

Identifying Polynucleotides or Nucleic Acids with Expression Libraries

In addition to hybridization methods, transcription factor homolog polypeptides can be obtained by screening an expression library using antibodies specific for one or more transcription factors. With the provision herein of the disclosed transcription factor, and transcription factor homolog nucleic acid sequences, the encoded polypeptide(s) can be expressed and purified in a heterologous expression system (e.g., E. coli) and used to raise antibodies (monoclonal or polyclonal) specific for the polypeptide(s) in question. Antibodies can also be raised against synthetic peptides derived from the amino acid sequences or subsequences of a transcription factor or transcription factor homolog. Methods of raising antibodies are well known in the art and are described in Harlow and Lane (1988), Antibodies: A Laboratory Manual, Cold Spring Harbor Laboratory, New York. Such antibodies can then be used to screen an expression library produced from the plant from which it is desired to clone additional transcription factor homologs, using the methods described above. The selected cDNAs can be confirmed by sequencing and enzymatic activity.

### Sequence Variations

It will readily be appreciated by those of skill in the art, that a significant variety of polynucleotide sequences are capable of encoding the transcription factors and transcription factor homolog polypeptides of the invention. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (i.e., peptides having some degree of equivalent or similar biological activity) but differ in sequence from the sequence shown in the Sequence Listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide that confers abiotic stress or biotic tolerance in a plant that is morphologically and developmentally similar to wild-type. Included within this definition are polymorphisms that may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides. It is expected that these distinctions from wild-type will be in residues other than the specific mutated residues encompassed by the present invention, although it is anticipated that conservative or similar substitutions of the mutated residues may allow the polypeptide to retain similar structural and functional roles in plants by conferring abiotic or biotic stress tolerance and morphological and developmental similarity to wild-type plants.

Allelic variant refers to any of two or more alternative forms of a gene occupying the same chromosomal locus. Allelic variation arises naturally through mutation, and may result in phenotypic polymorphism within populations. Gene mutations can be silent (i.e., no change in the encoded polypeptide) or may encode polypeptides having altered amino acid sequence. The term allelic variant is also used herein to denote a protein encoded by an allelic variant of a gene. Splice variant refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Those skilled in the art would recognize that, for example, G28, SEQ ID NO: 6, represents a single transcription factor; allelic variation and alternative splicing may be expected to occur. Allelic variants of SEQ ID NO: 5, encoding the sequence for SEQ ID NO: 6, can be cloned by probing cDNA or genomic libraries from different individual organisms according to standard procedures. Allelic variants of the DNA sequence shown in SEQ ID NO: 5, including those containing silent mutations and those in which mutations result in amino acid sequence changes, are within the scope of the present invention, as are proteins which are allelic variants of SEQ ID NO: 6. cDNAs generated from alternatively spliced mRNAs, which retain the properties of the transcription factor, are included within the scope of the present invention, as are polypeptides encoded by such cDNAs and mRNAs. Allelic variants and splice variants of these sequences can be cloned by probing cDNA or genomic libraries from different individual organisms or tissues according to standard procedures known in the art (e.g., U.S. Pat. No. 6,388,064).

Thus, in addition to the sequences set forth in the Sequence Listing, the invention also encompasses related nucleic acid molecules that are allelic or splice variants of the sequences of the invention, polynucleotides that encode orthologs, paralogs, variants, and fragments thereof that function in conferring abiotic or biotic stress tolerance in plants that are morphologically and developmentally similar to wild type, and include sequences that are complementary to any of the above nucleotide sequences. The invention also includes sequences that encode allelic or splice variants of the polypeptide sequences of the invention, orthologs, paralogs, variants, and fragments thereof that confer abiotic stress tolerance in plants that are morphologically and developmentally similar to wild type. Related nucleic acid molecules also include nucleotide sequences encoding a polypeptide comprising or consisting essentially of a substitution, modification, addition and/or deletion of one or more amino acid residues compared to the polypeptide sequences of the invention. Such related polypeptides may comprise, for example, additions and/or deletions of one or more N-linked or O-linked glycosylation sites, or an addition and/or a deletion of one or more cysteine residues.

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, e.g., site-directed mutagenesis, available in the art. Accordingly, any and all such variations of a sequence selected from the above table are a feature of the invention.

In addition to silent variations, other conservative variations that alter one, or a few amino acid residues in the encoded polypeptide, can be made without altering the function of the polypeptide, these conservative variants are, likewise, a feature of the invention.

For example, substitutions, deletions and insertions introduced into the sequences provided in the Sequence Listing, are also envisioned by the invention. Such sequence modifications can be engineered into a sequence by site-directed mutagenesis (Wu (ed.) Methods Enzymol. (1993) vol. 217, Academic Press) or the other methods noted below. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, e.g., a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made when it is desired to maintain the activity of the protein.

Similar substitutions are those in which at least one residue in the amino acid sequence has been removed and a different residue inserted in its place. Such substitutions generally are made when it is desired to maintain the activity of the protein. Substitutions that are less conservative can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

### Further Modifying Sequences of the Invention—Mutation/Forced Evolution

In addition to generating silent or conservative substitutions as noted, above, the present invention includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, e.g., according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel, supra, provides additional details on mutagenesis methods. Artificial forced evolution methods are described, for example, by Stemmer (1994) Nature 370: 389-391, Stemmer (1994) Proc. Natl. Acad. Sci. 91: 10747-10751, and U.S. Pat. Nos. 5,811,238, 5,837,500, and 6,242,568. Methods for engineering synthetic transcription factors and other polypeptides are described, for example, by Zhang et al. (2000) J. Biol. Chem. 275: 33850-33860, Liu et al. (2001) J. Biol. Chem. 276: 11323-11334, and Isalan et al. (2001) Nature Biotechnol. 19: 656-660. Many other mutation and evolution methods are also available and expected to be within the skill of the practitioner.

Similarly, chemical or enzymatic alteration of expressed nucleic acids and polypeptides can be performed by standard methods. For example, a sequence can be modified by addition of lipids, sugars, peptides, organic or inorganic compounds, by the inclusion of modified nucleotides or amino acids, or the like. For example, protein modification techniques are illustrated in Ausubel, supra. Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, e.g., using the sequences herein as a starting substrate for the various modification approaches.

For example, optimized coding sequence containing codons preferred by a particular prokaryotic or eukaryotic host can be used e.g., to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for Saccharomyces cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations which modify the sequence to facilitate cloning, processing and/or expression of the gene product. For example, alterations can be introduced using techniques which are well known in the art, e.g., site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP16 or GAL4 (Moore et al. (1998) Proc. Natl. Acad. Sci. 95: 376-381; Aoyama et al. (1995) Plant Cell 7: 1773-1785), peptides derived from bacterial sequences (Ma and Ptashne (1987) Cell 51: 113-119) and synthetic peptides (Giniger and Ptashne (1987) Nature 330: 670-672).

### Expression and Modification of Polypeptides

Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homolog.

The transgenic plants of the present invention comprising recombinant polynucleotide sequences are generally derived from parental plants, which may themselves be non-transformed (or non-transgenic) plants. These transgenic plants may either have a transcription factor gene “knocked out” (for example, with a genomic insertion by homologous recombination, an antisense or ribozyme construct) or expressed to a normal or wild-type extent. However, overexpressing transgenic “progeny” plants will exhibit greater mRNA levels, wherein the mRNA encodes a transcription factor, that is, a DNA-binding protein that is capable of binding to a DNA regulatory sequence and inducing transcription, and preferably, expression of a plant trait gene, such as a gene that increases abiotic or biotic stress tolerance. Preferably, the mRNA expression level will be at least three-fold greater than that of the parental plant, or more preferably at least ten-fold greater mRNA levels compared to said parental plant, and most preferably at least fifty-fold greater compared to said parental plant.

### Vectors, Promoters, and Expression Systems

The present invention includes recombinant constructs comprising one or more of the nucleic acid sequences herein. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts that describe molecular biological techniques useful herein, including the use and production of vectors, promoters and many other relevant topics, include Berger, supra, Sambrook, supra and Ausubel, supra. Any of the identified sequences can be incorporated into a cassette or vector, e.g., for expression in plants (in this case, the cassette or vector is thus an “expression cassette” or “expression vector”). A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucleic Acids Res. 12: 8711-8721, Klee (1985) Bio/Technology 3: 637-642, for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) Bio/Technology 9: 957-962) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol. 102: 1077-1084; Vasil (1993) Bio/Technology 10: 667-674; Wan and Lemeaux (1994) Plant Physiol. 104: 37-48, and for Agrobacterium-mediated DNA transfer (Ishida et al. (1996) Nature Biotechiol. 14: 745-750).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

A potential utility for the transcription factor polynucleotides disclosed herein is the isolation of promoter elements from these genes that can be used to program expression in plants of any genes. Each transcription factor gene disclosed herein is expressed in a unique fashion, as determined by promoter elements located upstream of the start of translation, and additionally within an intron of the transcription factor gene or downstream of the termination codon of the gene. As is well known in the art, for a significant portion of genes, the promoter sequences are located entirely in the region directly upstream of the start of translation. In such cases, typically the promoter sequences are located within 2.0 kb of the start of translation, or within 1.5 kb of the start of translation, frequently within 1.0 kb of the start of translation, and sometimes within 0.5 kb of the start of translation.

The promoter sequences can be isolated according to methods known to one skilled in the art.

Examples of constitutive plant promoters which can be useful for expressing the TF sequence include: the cauliflower mosaic virus (CaMV) 35S promoter, which confers constitutive, high-level expression in most plant tissues (e.g., Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

The transcription factors of the invention may be operably linked with a specific promoter that causes the transcription factor to be expressed in response to environmental, tissue-specific or temporal signals. A variety of plant gene promoters that regulate gene expression in response to environmental, hormonal, chemical, developmental signals, and in a tissue-active manner can be used for expression of a TF sequence in plants. Choice of a promoter is based largely on the phenotype of interest and is determined by such factors as tissue (e.g. i seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening, such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11: 651-662), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli and Keller (1998) Plant Mol. Biol. 37: 977-988), flower-specific (Kaiser et al. (1995) Plant Mol. Biol. 28: 231-243), pollen (Baerson et al. (1994) Plant Mol. Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al., (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) Plant Molec. Biol. 38: 817-825) and the like. Additional promoters are those that elicit expression in response to heat (Ainley et al. (1993) Plant Mol. Biol. 22: 13-23), light (e.g., the pea rbcS-3A promoter, Kuhlemeier et al. (1989) Plant Cell 1: 471-478, and the maize rbcS promoter, Schaffner and Sheen (1991) Plant Cell 3: 997-1012); wounding (e.g., wunI, Siebertz et al. (1989) Plant Cell 1: 961-968); pathogens (such as the PR-1 promoter described in Buchel et al. (1999) Plant Mol. Biol. 40: 387-396, and the PDF1.2 promoter described in Manners et al. (1998) Plant Mol. Biol. 38: 1071-1080), and chemicals such as methyl jasmonate or salicylic acid (Gatz (1997) Annu. Rev. Plant Physiol. Plant Mol. Biol. 48: 89-108). In addition, the timing of the expression can be controlled by using promoters such as those acting at senescence (Gan and Amasino (1995) Science 270: 1986-1988); or late seed development (Odell et al. (1994) Plant Physiol. 106: 447-458).

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

### Additional Expression Elements

Specific initiation signals can aid in efficient translation of coding sequences. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon can be separately provided. The initiation codon is provided in the correct reading frame to facilitate transcription. Exogenous transcriptional elements and initiation codons can be of various origins, both natural and synthetic. The efficiency of expression can be enhanced by the inclusion of enhancers appropriate to the cell system in use.

### Expression Hosts

The present invention also relates to host cells which are transduced with vectors of the invention, and the production of polypeptides of the invention (including fragments thereof) by recombinant techniques. Host cells are genetically engineered (i.e., nucleic acids are introduced, e.g., transduced, transformed or transfected) with the vectors of this invention, which may be, for example, a cloning vector or an expression vector comprising the relevant nucleic acids herein. The vector is optionally a plasmid, a viral particle, a phage, a naked nucleic acid, etc. The engineered host cells can be cultured in conventional nutrient media modified as appropriate for activating promoters, selecting transformants, or amplifying the relevant gene. The culture conditions, such as temperature, pH and the like, are those previously used with the host cell selected for expression, and will be apparent to those skilled in the art and in the references cited herein, including, Sambrook, supra and Ausubel, supra.

The host cell can be a eukaryotic cell, such as a yeast cell, or a plant cell, or the host cell can be a prokaryotic cell, such as a bacterial cell. Plant protoplasts are also suitable for some applications. For example, the DNA fragments are introduced into plant tissues, cultured plant cells or plant protoplasts by standard methods including electroporation (Fromm et al. (1985) Proc. Natl. Acad. Sci. 82: 5824-5828, infection by viral vectors such as cauliflower mosaic virus (CaMV) (Hohn et al. (1982) Molecular Biology of Plant Tumors Academic Press, New York, N.Y., pp. 549-560; U.S. Pat. No. 4,407,956), high velocity ballistic penetration by small particles with the nucleic acid either within the matrix of small beads orparticles, or on the surface (Klein et al. (1987) Nature 327: 70-73), use of pollen as vector (WO 85/01856), or use of Agrobacterium tumefaciens or A. rhizogenes carrying aT-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233: 496-498; Fraley et al. (1983) Proc. Natl. Acad. Sci. 80: 4803-4807).

The cell can include a nucleic acid of the invention that encodes a polypeptide, wherein the cell expresses a polypeptide of the invention. The cell can also include vector sequences, or the like. Furthermore, cells and transgenic plants that include any polypeptide or nucleic acid above or throughout this specification, e.g., produced by transduction of a vector of the invention, are an additional feature of the invention.

For long-term, high-yield production of recombinant proteins, stable expression can be used. Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding mature proteins of the invention can be designed with signal sequences which direct secretion of the mature polypeptides through a prokaryotic or eukaryotic cell membrane.

### Modified Amino Acid Residues

Polypeptides of the invention may contain one or more modified amino acid residues. The presence of modified amino acids may be advantageous in, for example, increasing polypeptide half-life, reducing polypeptide antigenicity or toxicity, increasing polypeptide storage stability, or the like. Amino acid residue(s) are modified, for example, co-translationally or post-translationally during recombinant production or modified by synthetic or chemical means.

Non-limiting examples of a modified amino acid residue include incorporation or other use of acetylated amino acids, glycosylated amino acids, sulfated amino acids, prenylated (e.g., farnesylated, geranylgeranylated) amino acids, PEG modified (e.g., “PEGylated”) amino acids, biotinylated amino acids, carboxylated amino acids, phosphorylated amino acids, etc. References adequate to guide one of skill in the modification of amino acid residues are replete throughout the literature.

The modified amino acid residues may prevent or increase affinity of the polypeptide for another molecule, including, but not limited to, polynucleotide, proteins, carbohydrates, lipids and lipid derivatives, and other organic or synthetic compounds.

### Production of Transgenic Plants

### Modification of Traits

The polynucleotides of the invention are used to produce transgenic plants with various traits, or characteristics that have been modified in a desirable manner, e.g., to improve the abiotic or biotic stress tolerance characteristics of a plant. For example, alteration of expression levels or patterns (e.g., spatial or temporal expression patterns) of one or more of the transcription factors (or transcription factor homologs) of the invention, as compared with the levels of the same protein found in a wild-type plant, can be used to modify a plant's traits. An illustrative example of trait modification, improved characteristics, by altering expression levels of a particular transcription factor is described further in the Examples and the Sequence Listing.

### Arabidopsis as a Model System

Arabidopsis thaliana is the object of rapidly growing attention as a model for genetics and metabolism in plants. Arabidopsis has a small genome, and well-documented studies are available. It is easy to grow in large numbers and mutants defining important genetically controlled mechanisms are either available, or can readily be obtained. Various methods to introduce and express isolated homologous genes are available (e.g., Koncz et al., eds., Methods in Arabidopsis Research (1992) World Scientific, New Jersey, N.J., in “Preface”). Because of its small size, short life cycle, obligate autogamy and high fertility, Arabidopsis is also a choice organism for the isolation of mutants and studies in morphogenetic and development pathways, and control of these pathways by transcription factors (Koncz (1992) supra, p. 72). A number of studies introducing transcription factors into A. thaliana have demonstrated the utility of this plant for understanding the mechanisms of gene regulation and trait alteration in plants (for example, in Koncz (1992) supra, and U.S. Pat. No. 6,417,428).

Homologous Genes Introduced into Transgenic Plants.

Homologous genes that may be derived from any plant, or from any source whether natural, synthetic, semi-synthetic or recombinant, and that share significant sequence identity or similarity to those provided by the present invention, may be introduced into plants, for example, crop plants, to confer desirable or improved traits. Consequently, transgenic plants may be produced that comprise a recombinant expression vector or cassette with a promoter operably linked to one or more sequences homologous to presently disclosed sequences. The promoter may be, for example, a plant or viral promoter.

The invention thus provides for methods for preparing transgenic plants, and for modifying plant traits. These methods include introducing into a plant a recombinant expression vector or cassette comprising a functional promoter operably linked to one or more sequences homologous to presently disclosed sequences. Plants and kits for producing these plants that result from the application of these methods are also encompassed by the present invention.

Genes Traits and Utilities that Affect Plant Characteristics

Plant transcription factors can modulate gene expression, and, in turn, be modulated by the environmental experience of a plant. Significant alterations in a plant's environment invariably result in a change in the plant's transcription factor gene expression pattern. Altered transcription factor expression patterns generally result in phenotypic changes in the plant. Transcription factor gene product(s) in transgenic plants then differ(s) in amounts or proportions from that found in wild-type or non-transformed plants, and those transcription factors likely represent polypeptides that are used to alter the response to the environmental change. By way of example, it is well accepted in the art that analytical methods based on altered expression patterns may be used to screen for phenotypic changes in a plant far more effectively than can be achieved using traditional methods.

Potential Applications of the Presently Disclosed Sequences that Regulate Abiotic or Biotic Stress Tolerance

The genes identified by the presently disclosed experiments represent potential regulators of responses to abiotic stress. As such, these genes (or their orthologs and paralogs) could be applied to commercial species in order to improve yield, and potentially allow certain crops to be grown under conditions of hyperosmotic (e.g., drought, freezing, high salinity) or other abiotic stresses (e.g., heat, cold), or when challenged by a plant pathogen.

Arabidopsis plants that overexpress the AP2 transcriptional activators can be stunted in their growth and delayed in flowering, e.g., when the activators are expressed at high levels. As noted in the Examples below, it is possible that mutant versions of some genes suppress the potentially “negative” traits associated with transcription factor polypeptide overexpression (e.g., stunted and delayed flowering phenotypes), but retain the positive effects of improved stress tolerance that are conferred by AP2 polypeptide overexpression. Such mutants have now been identified and characterized, as shown in the below Examples. These altered AP2 genes can be used to improve stress tolerance of plants with fewer or reduced adverse secondary effects on plant growth and development. By way of a further improvement, regulation of these modified genes with tissue-specific or inducible promoters, for example, stress-inducible promoters, could provide increased tolerance to environmental stresses without significantly impacting a plant's phenotype in a negative manner, such as by decreasing seed production, reducing plant size, and/or delaying flowering.

### EXAMPLES

The invention, now being generally described, will be more readily understood by reference to the following examples, which are included merely for purposes of illustration of certain aspects and embodiments of the present invention and are not intended to limit the invention. It will be recognized by one of skill in the art that a transcription factor that is associated with a particular first trait may also be associated with at least one other, unrelated and/or inherent second trait that was not predicted by the first trait.

### Example I

### Full Length Gene Identification and Cloning

Putative transcription factor sequences (genomic or ESTs) related to known transcription factors were identified in the Arabidopsis thaliana GenBank database using the tblastn sequence analysis program using default parameters and a P-value cutoff threshold of −4 or −5 or lower, depending on the length of the query sequence. Putative transcription factor sequence hits were then screened to identify those containing particular sequence strings. If the sequence hits contained such sequence strings, the sequences were confirmed as transcription factors.

Alternatively, Arabidopsis thaliana cDNA libraries derived from different tissues or treatments, or genomic libraries were screened to identify novel members of a transcription family using a low stringency hybridization approach. Probes were synthesized using gene specific primers in a standard PCR reaction (annealing temperature 60° C.) and labeled with ³²P dCTP using the High Prime DNA Labeling Kit (Boehringer Mannheim Corp. (now Roche Diagnostics Corp., Indianapolis, Ind.). Purified radiolabelled probes were added to filters immersed in Church hybridization medium (0.5 M NaPO₄, pH 7.0, 7% SDS, 1% w/v bovine serum albumin) and hybridized overnight at 60° C. with shaking. Filters were washed two times for 45 to 60 minutes with 1×SCC, 1% SDS at 60° C.

To identify additional sequence 5′ or 3′ of a partial cDNA sequence in a cDNA library, 5′ and 3′ rapid amplification of cDNA ends (RACE) was performed using the MARATHON cDNA amplification kit (Clontech, Palo Alto, Calif.). Generally, the method entailed first isolating poly(A) mRNA, performing first and second strand cDNA synthesis to generate double stranded cDNA, blunting cDNA ends, followed by ligation of the MARATHON Adaptor to the cDNA to form a library of adaptor-ligated ds cDNA.

Gene-specific primers were designed to be used along with adaptor specific primers for both 5′ and 3′ RACE reactions. Nested primers, rather than single primers, were used to increase PCR specificity. Using 5′ and 3′ RACE reactions, 5′ and 3′ RACE fragments were obtained, sequenced and cloned. The process can be repeated until 5′ and 3′ ends of the full-length gene were identified. Then the full-length cDNA was generated by PCR using primers specific to 5′ and 3′ ends of the gene by end-to-end PCR.

### Example II

### In Vitro Mutagenesis

One method that may be used to create a transgenic plant overexpressing a modified transcription factor may be with the use of physical or chemical mutagenizing agents that may be used directly on isolated DNA. For example, an isolated AP2 polynucleotide sequence may be subjected to UV irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, or nucleotide analogues. When such agents are used, the mutagenesis is typically performed by incubating the DNA sequence encoding a transcription factor to be mutagenized in the presence of the mutagenizing agent of choice under suitable conditions well known in the art. The DNA may then be incorporated into a vector and transformed into a plant cell, which may then be regenerated into a plant. It may be desirable to amplify the mutated DNA (for example, using PCR) prior to insertion into the vector.

Another alternative mutagenesis method is PCR-generated mutagenesis, in which a chemically treated or non-treated gene encoding a transcription factor is subjected to PCR under conditions that increase the misincorporation of nucleotides (for example, in Leung et al., (1989) Technique, 1: 11-15; and Deshler (1992) Genet Anal. Tech. Appl. 9: 103-106)

### Example III

### Construction of Expression Vectors

The sequence was amplified from a genomic or cDNA library using primers specific to sequences upstream and downstream of the coding region. The expression vector was pMEN20 or pMEN65, which are both derived from pMON316 (Sanders et al. (1987) Nucleic Acids Res. 15:1543-1558) and contain the CaMV 35S promoter to express transgenes (pMEN20 is an earlier version of pMEN65 in which the kanamycin resistance gene is driven by the 35S promoter rather than the nos promoter). To clone the sequence into the vector, both pMEN20 and the amplified DNA fragment were digested separately with SalI and NotI restriction enzymes at 37° C. for 2 hours. The digestion products were subject to electrophoresis in a 0.8% agarose gel and visualized by ethidium bromide staining. The DNA fragments containing the sequence and the linearized plasmid were excised and purified by using a QIAQUICK gel extraction kit (Qiagen, Valencia, Calif.). The fragments of interest were ligated at a ratio of 3:1 (vector to insert). Ligation reactions using T4 DNA ligase (New England Biolabs, Beverly Mass.) were carried out at 16° C. for 16 hours. The ligated DNAs were transformed into competent cells of the E. coli strain DH5alpha by using the heat shock method. The transformations were plated on LB plates containing 50 mg/l kanamycin (Sigma Chemical Co. St. Louis Mo.). Individual colonies were grown overnight in five milliliters of LB broth containing 50 mg/l kanamycin at 37° C. Plasmid DNA was purified by using Qiaquick Mini Prep kits (Qiagen, Valencia, Calif.).

Two-component vectors. Two-component base vectors were used to express genes under the control of the LexA operator. They each contain eight tandem LexA operators from plasmid p8op-lacZ (Clontech) followed by a polylinker. The plasmid carries a sulfonamide resistance gene driven by the 35S promoter.

GAL4 fusion vectors. Backbone vector for creation of N-terminal GAL4 activation domain protein fusions were created by inserting the GAL4 activation domain into the BglII and KpnI sites of pMEN65. To create gene fusions, the transcription factor gene of interest is amplified using a primer that starts at the second amino acid and has added the KpnI or SalI and NotI sites. The PCR product is then cloned into the KpnI or SalI and NotI sites of P21195, taking care to maintain the reading frame.

Backbone vectors for creation of C-terminal GALA activation domain fusions were constructed by amplification of the GALA activation domain and insertion of this domain into the NotI and XbaI sites of pMEN65. To create gene fusions, the transcription factor gene of interest was amplified using a 3′ primer that ends at the last amino acid codon before the stop codon. The PCR product was be cloned into the SalI and NotI sites.

A derivative of pMEN20 that carries a CBF1:GAL4 fusion was used to construct other GAL4 fusions. In this method, the CBF1 gene was removed with SalI or KpnI and EcoRI. The gene of interest was amplified using a 3′ primer that ended at the last amino acid codon before the stop codon and contained an EcoRI or Mfe1 site. The product was inserted into these SalI or KpnI and EcoRI sites, taking care to maintain the reading frame.

These method steps may also be used to construct expression vectors harboring AP2 gene mutations created in vitro as described in Example II.

### Example IV

### Protein Variants

A variety of other methods for generating variations of native proteins can be envisioned and, in several cases noted below, have been generated, including adding domains using a recombinant approach, or deleting regions of the nucleotide sequence selectively or randomly. These methods may result in an altered polypeptide product by virtue of, for example, a reading frame shift, alternative splicing of transcripts, or a sizeable deletion or addition to the polypeptide that results in an altered function or binding affinity. Following routine propagation and/or crossing methods, plant populations that are homozygous or heterozygous for mutations of interest may be generated. Tissue from these plants may be harvested for nucleic acid isolation, analysis, and as a source for the mutation used in subsequent studies.

Examples of constructs harboring variations in G912, G1792, G28, G867 and G47 (SEQ ID NOs: 2, 4, 6, 8 and 10, respectively) are listed below. Each of the following constructs carried a kanamycin resistance marker. Morphological and stress results obtained for plants overexpressing these AP2 variants are noted in Example XV.

P21270 (SEQ ID NO: 16) is an overexpression construct encoding a truncated version of the G912 protein, comprising only the AP2 domain (amino acid coordinates 51-118 of G912) and the two CBF boxes (amino acid coordinates 38-52 and 107-113, or PKKRAGRKKFRETRHP and LNFADSAWR of G912, for Box I and Box II, respectively). This truncated version of the G912 protein was overexpressed in Arabidopsis. Interestingly, these plants showed no obvious differences in morphology compared to wild-type controls. Thus, the regions of the G912 protein that caused undesirable morphologies are likely external to the AP2 and CBF Box I/II domains.

P21194 (SEQ ID NO: 17) is an overexpression construct encoding a G912 clone that has a GAL4 transactivation domain fused at the C terminus (35S::G912-GAL4).

P21197 (SEQ ID NO: 18) is an overexpression construct encoding a G912 clone that has a GAL4 transactivation domain fused at the N terminus (35S::GAL4-G912). The aim of the latter two projects was to determine whether the efficacy of the G912 protein could be improved by addition of an artificial GAL4 activation domain.

P25437 (SEQ ID NO: 19) is an overexpression construct carrying a truncated form of G1792 that comprises only the first 115 amino acids: 35S::G1792(aa1-115).

The following overexpression constructs containing site-directed mutagenized clones have been made. Each construct contains a 35S direct promoter fusion to the mutagenized G1792 clone, and carries KanR.

P25739 (SEQ ID NO: 20) 35S::G1792(D124G); P25740 (SEQ ID NO: 21) 35S::G1792(L128G); and P25741 (SEQ ID NO: 22) 35S::G1792(L132G).

P25083 (SEQ ID NO: 23) comprises a 35S::G1792-GALA direct promoter fusion.

P25093 (SEQ ID NO: 24) comprises a 35S::GAL4-G1792 direct promoter fusion The aim of the latter two projects was to determine whether the efficacy of the G1792 protein could be improved by addition of an artificial GAL4 activation domain.

P25271 (SEQ ID NO: 25) carries a 35S::G1792-green fluorescent protein (GFP) fusion directly fused to a ³⁵S promoter. These lines could have a variety of applications including analyses to determine sub-cellular localization of the transcription factor protein.

The following overexpression constructs containing site-directed mutagenized clones contain a 35S direct promoter fusion to the mutagenized G28 clone, and carries KanR.

P25678 (SEQ ID NO: 26) 35S::G28(T65D); P25680 (SEQ ID NO: 27) 35S::G28(T180D); P25682 (SEQ ID NO: 28) 35S::G28(T65D,T180D); and P25684 (SEQ ID NO: 29) 35S::G28(T65D,T177D,T180D).

P21143 (SEQ ID NO: 30) contains a 35S::G28-GAL4 fusion.

P21196 (SEQ ID NO: 31) comprises a 35S::GALA-G28 direct promoter fusion. The aim of the latter two projects was to determine whether the efficacy of the G28 protein could be improved by addition of an artificial GAL4 activation domain.

P21276 (SEQ ID NO: 32) is an overexpression construct encoding a truncated version of the G867 protein (residues 139 to 309 from the wild-type protein) containing the B3 domain but not the AP2 domain.

P21275 (SEQ ID NO: 33) is an overexpression construct encoding a truncated version of the G867 protein containing only the AP2 domain (residues 36 to 165 of the wild-type protein).

P21193 (SEQ ID NO: 34) is an overexpression construct encoding a G867 clone that has a GALA transactivation domain fused at the C terminus (35S::G867-GAL4).

P21201 (SEQ ID NO: 35) is an overexpression construct encoding a G867 clone that has a GALA transactivation domain fused at the N terminus (35S::GAL4-G867).

P25301 (SEQ ID NO: 36) carries a 35S::G867-GFP fusion directly fused to the 35S promoter.

The following overexpression constructs containing site-directed mutagenized clones have been made. Each construct contains a 35S direct promoter fusion to the mutagenized G47 clone, and carries KanR.

P25732 (SEQ ID NO: 37): 35S::G47(V31E); P25733 (SEQ ID NO: 38): 35S::G47(V51R); and P25735: (SEQ ID NO: 39) 35S::G47(V55T).

P25186 (SEQ ID NO: 40) is an overexpression construct encoding a G47 clone that has a GAL4 transactivation domain fused at the N terminus (35S::GALA-G47).

P25279 (SEQ ID NO: 41) carries a 35S::G47-GFP fusion directly fused to the 35S promoter and a KanR marker.

### Example V

### Transformation of Agrobacterium with the Expression Vector

After the expression constructs were generated, the constructs were used to transform Agrobacterium tumefaciens cells expressing the gene products. The stock of Agrobacteriun tumefaciens cells for transformation was made as described by Nagel et al. (1990) FEMS Microbiol Letts. 67: 325-328. Agrobacterium strain ABI was grown in 250 ml LB medium (Sigma) overnight at 28° C. with shaking until an absorbance over 1 cm at 600 nm (A₆₀₀) of 0.5-1.0 was reached. Cells were harvested by centrifugation at 4,000×g for 15 min at 4° C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with constructs prepared as described above following the protocol described by Nagel et al. (supra). For each DNA construct to be transformed, 50-100 ng DNA (generally resuspended in 10 mM Tris-HCl, 1 mM EDTA, pH 8.0) were mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells were immediately resuspended in 1.0 ml LB and allowed to recover without antibiotic selection for 2-4 hours at 28° C. in a shaking incubator. After recovery, cells were plated onto selective medium of LB broth containing 100 μg/ml spectinomycin (Sigma) and incubated for 24-48 hours at 28° C. Single colonies were then picked and inoculated in fresh medium. The presence of the plasmid construct was verified by PCR amplification and sequence analysis.

### Example VI

### Transformation of Arabidopsis Plants with Agrobacterium tumefaciens

After transformation of Agrobacterium tumefaciens with the constructs or plasmid vectors containing the gene of interest, single Agrobacterium colonies were identified, propagated, and used to transform Arabidopsis plants. Briefly, 500 ml cultures of LB medium containing 50 mg/l kanamycin were inoculated with the colonies and grown at 28° C. with shaking for 2 days until an optical absorbance at 600 nm wavelength over 1 cm (A₆₀₀) of >2.0 is reached. Cells were then harvested by centrifugation at 4,000×g for 10 min, and resuspended in infiltration medium (½× Murashige and Skoog salts (Sigma), 1× Gamborg's B-5 vitamins (Sigma), 5.0% (w/v) sucrose (Sigma), 0.044 μM benzylamino purine (Sigma), 200 μl/l Silwet L-77 (Lehle Seeds) until an A₆₀₀ of 0.8 was reached.

Prior to transformation, Arabidopsis thaliana seeds (ecotype Columbia) were sown at a density of ˜10 plants per 4″ pot onto Pro-Mix BX potting medium (Hummert International) covered with fiberglass mesh (18 mm×16 mm). Plants were grown under continuous illumination (50-75 μE/m²/sec) at 22-23° C. with 65-70% relative humidity. After about 4 weeks, primary inflorescence stems (bolts) are cut off to encourage growth of multiple secondary bolts. After flowering of the mature secondary bolts, plants were prepared for transformation by removal of all siliques and opened flowers.

The pots were then immersed upside down in the mixture of Agrobacterium infiltration medium as described above for 30 sec, and placed on their sides to allow draining into a 1′×2′ flat surface covered with plastic wrap. After 24 h, the plastic wrap was removed and pots are turned upright. The immersion procedure was repeated one week later, for a total of two immersions per pot. Seeds were then collected from each transformation pot and analyzed following the protocol described below.

### Example VII

### Identification of Arabidopsis Primary Transformants

Seeds collected from the transformation pots were sterilized essentially as follows. Seeds were dispersed into in a solution containing 0.1% (v/v) Triton X-100 (Sigma) and sterile water and washed by shaking the suspension for 20 min. The wash solution was then drained and replaced with fresh wash solution to wash the seeds for 20 min with shaking. After removal of the ethanol/detergent solution, a solution containing 0.1% (v/v) Triton X-100 and 30% (v/v) bleach (CLOROX; Clorox Corp. Oakland Calif.) was added to the seeds, and the suspension was shaken for 10 min. After removal of the bleach/detergent solution, seeds were then washed five times in sterile distilled water. The seeds were stored in the last wash water at 4° C. for 2 days in the dark before being plated onto antibiotic selection medium (1× Murashige and Skoog salts (pH adjusted to 5.7 with 1M KOH), 1× Gamborg's B-5 vitamins, 0.9% phytagar (Life Technologies), and 50 mg/l kanamycin). Seeds were germinated under continuous illumination (50-75 μE/m²/sec) at 22-23° C. After 7-10 days of growth under these conditions, kanamycin resistant primary transformants (T1 generation) were visible and obtained. These seedlings were transferred first to fresh selection plates where the seedlings continued to grow for 3-5 more days, and then to soil (Pro-Mix BX potting medium).

Primary transformants were crossed and progeny seeds (T₂) collected; kanamycin resistant seedlings were selected and analyzed. The expression levels of the recombinant polynucleotides in the transformants vary from about a 5% expression level increase to a least a 100% expression level increase. Similar observations are made with respect to polypeptide level expression.

### Example VIII

### Identification of Modified Phenotypes in Overexpressing Arabidopsis Plants

Experiments were performed to identify those transformants that exhibited stress-tolerant phenotypes and few, if any, morphological differences relative to wild-type control plants, i.e., a modified structure, physiology, and/or development characteristics. For such studies, the transformants were exposed to various assay conditions and novel structural, physiological responses, or developmental characteristics associated with the ectopic expression of the polynucleotides or polypeptides of the invention were observed. Examples of genes and equivalogs that confer significant improvements to overexpressing plants are noted.

Experiments were also performed to identify those transformants that exhibited an improved pathogen tolerance. The goal of these experiments was to determine if disease resistance could be achieved while reducing detrimental pleiotropic effects of ectopic- or over-expression. To test the spectrum of resistance, assays were performed for Botrytis cinerea, Fusarium oxysporum, Erysiphe orontii and Sclerotinia sclerotiorum.

Having established a homozygous population carrying each construct, it was then possible to overexpress any transcription factor of the invention by super-transforming or crossing in a second construct (opLexA::transcription factor) carrying the transcription factor of interest cloned behind a LexA operator site. In each case this second construct carried a sulfonamide selectable marker and was contained within vector backbone.

A number of lines were selected for plate-based disease assays. Included in the disease assays were challenges by one of a number of diverse fungal pathogens. T1 or T2 seeds from each line (segregating for the target transgene construct) were surface sterilized and grown on MS plates supplemented with 0.3% sucrose. Plants homozygous for each activator line and supertransformed with the target construct vector containing GUS (no transcription factor gene) were used as controls and treated in the same manner as test lines. Plants were grown in a 22° C. growth chamber under constant light for ten days. On the tenth day, seedlings were transferred to MS plates without sucrose. Each plate was marked with half of the plate containing nine seedlings of an experimental line and the other half containing nine seedlings of the control line. For each experimental line, there were three test plates per pathogen plus one uninoculated plate. Lines that constitutively expressed the sequences of the invention under the direct control of the 35S promoter were included and compared to wild-type plants as a control for the disease assays. Direct 35S/gene fusion lines were also used in the abiotic stress assay experiments, for which results are presented in Table 2.

At 14 days, seedlings were inoculated by spraying the plates with a freshly prepared suspension of spores (10⁵ spores/ml, Botrytis; 10⁶ spores/ml, Fusarium) or ground, filtered hyphae (1 gm/300 ml, Sclerotinia). Plates were returned to a growth chamber with dimmed lighting on a 12 hour dark/12 hour light regimen; disease symptoms were assessed over a period of two weeks after inoculation. All lines were initially tested with Botrytis and Sclerotinia. Tolerance was quantitatively scored as the number of living plants. Numbers were plotted on a “box and whisker” diagram to determine increased survivorship of particular promoter/gene combinations. To illustrate the spread of the data, results from all lines per combination were plotted together; lines that were potentially sense-suppressed (based on disease phenotype) may skew the median towards wild type in some cases. Also, all two-component lines were segregating for the target transgene. Lines that showed tolerance to Botrytis or Sclerotinia were then tested with Fusarium. Fusarium tolerance was determined by a reduction in chlorosis and damping off symptoms.

A number of plant lines overexpressing some of the G1792 or G28 clade members were tested in a soil-based assay for resistance to powdery mildew (Erysiphe cichoracearum). Typically, eight lines per project were subjected to the Erysiphe assay. Erysiphe cichoracearum inoculum was propagated on a pad4 mutant line in the Col-0 background, which is highly susceptible to Erysiphe (Reuber et al. (1998) Plant J. 16: 473-485). The inocula were maintained by using a small paintbrush to dust conidia from a 2-3 week old culture onto new plants (generally three weeks old). For the assay, seedlings were grown on plates for one week under 24-hour light in a germination chamber, then transplanted to soil and grown in a walk-in growth chamber under a 12-hour light/12-hour dark light regimen, 70% humidity. Each line was transplanted to two 13 cm square pots, nine plants per pot. In addition, three control plants were transplanted to each pot for direct comparison with the test line. Approximately 3.5 weeks after transplanting, plants were inoculated using settling towers, as described by Reuber et al. (1998) supra. Generally, three to four heavily infested leaves were used per pot for the disease assay. Level of fungal growth was evaluated eight to ten days after inoculation.

Assays were also performed to identify those transformants that exhibited improved abiotic stress tolerance. The germination assays followed modifications of the same basic protocol. Sterile seeds were sown on the conditional media listed below. Plates were incubated at 22° C. under 24-hour light (120-130 lEin/m²/s) in a growth chamber. Evaluation of germination and seedling vigor was conducted 3 to 15 days after planting. The basal media was 80% Murashige-Skoog medium (MS)+vitamins.

For abiotic stress experiments conducted with seedlings, seeds were germinated and grown for seven days on MS+vitamins+1% sucrose at 22° C. and then transferred to cold and heat stress conditions. The plants were either exposed to cold stress (6 hour exposure to 8° C.), or heat stress (32° C. was applied for five days, after which the plants were transferred back 22° C. for recovery and evaluated after 5 days relative to controls not exposed to the depressed or elevated temperature).

Salt stress assays were intended to find genes that confer better germination, seedling vigor or growth in high salt. Evaporation from the soil surface causes upward water movement and salt accumulation in the upper soil layer where the seeds are placed. Thus, germination normally takes place at a salt concentration much higher than the mean salt concentration of the whole soil profile. Plants differ in their tolerance to NaCl depending on their stage of development, therefore seed germination, seedling vigor, and plant growth responses were evaluated.

Hyperosmotic stress assays (including NaCl and mannitol assays) were conducted to determine if an osmotic stress phenotype was NaCl-specific or if it was a general hyperosmotic stress related phenotype. Plants tolerant to hyperosmotic stress could also have more tolerance to drought and/or freezing.

For salt and hyperosmotic stress germination experiments, the medium was supplemented with 150 mM NaCl or 300 mM mannitol. Growth regulator sensitivity assays were performed in MS media, vitamins, and either 0.3 μM ABA 9.4% sucrose, or 5% glucose.

Desiccation and drought assays were performed to find genes that mediate better plant survival after short-term, severe water deprivation. Ion leakage was measured if needed.

For plate-based desiccation assays, wild-type and control seedlings were grown for 14 days on MS+Vitamins+1% Sucrose at 22° C. The plates were then left open in the sterile hood for 3 hr for hardening, and the seedlings were removed from the media and dried for 1.5 h in the sterile hood. The seedlings were transferred back to plates and incubated at 22° C. for recovery. The plants were then evaluated after another five days.

Soil-based drought screens were performed with Arabidopsis plants overexpressing the transcription factors listed in the Sequence Listing, where noted below. Seeds from wild-type Arabidopsis plants, or plants overexpressing a polypeptide of the invention, were stratified for three days at 4° C. in 0.1% agarose. Fourteen seeds of each overexpressor or wild-type were then sown in three inch clay pots containing a 50:50 mix of vermiculite:perlite topped with a small layer of MetroMix 200 and grown for fifteen days under 24 hr light. Pots containing wild-type and overexpressing seedlings were placed in flats in random order. Drought stress was initiated by placing pots on absorbent paper for seven to eight days. The seedlings were considered to be sufficiently stressed when the majority of the pots containing wild-type seedlings within a flat had become severely wilted. Pots were then re-watered and survival was scored four to seven days later. Plants were ranked against wild-type controls for each of two criteria: tolerance to the drought conditions and recovery (survival) following re-watering.

At the end of the initial drought period, each pot was assigned a numeric value score depending on the above criteria. A low value was assigned to plants with an extremely poor appearance (i.e., the plants were uniformly brown) and a high value given to plants that were rated very healthy in appearance (i.e., the plants were all green). After the plants were rewatered and incubated an additional four to seven days, the plants were reevaluated to indicate the degree of recovery from the water deprivation treatment.

An analysis was then conducted to determine which plants best survived water deprivation, identifying the transgenes that consistently conferred drought-tolerant phenotypes and their ability to recover from this treatment. The analysis was performed by comparing overall and within flat tabulations with a set of statistical models to account for variations between batches. Several measures of survival were tabulated, including: (a) the average proportion of plants surviving relative to wild-type survival within the same flat; (b) the median proportion surviving relative to wild-type survival within the same flat; (c) the overall average survival (taken over all batches, flats, and pots); (d) the overall average survival relative to the overall wild-type survival; and (e) the average visual score of plant health before rewatering.

Sugar sensing assays were intended to find genes involved in sugar sensing by germinating seeds on high concentrations of sucrose and glucose and looking for degrees of hypocotyl elongation. The germination assay on mannitol controlled for responses related to osmotic stress. Sugars are key regulatory molecules that affect diverse processes in higher plants including germination, growth, flowering, senescence, sugar metabolism and photosynthesis. Sucrose is the major transport form of photosynthate and its flux through cells has been shown to affect gene expression and alter storage compound accumulation in seeds (source-sink relationships). Glucose-specific hexose-sensing has also been described in plants and is implicated in cell division and repression of “famine” genes (photosynthetic or glyoxylate cycles).

Temperature stress assays were carried out to find genes that confer better germination, seedling vigor or plant growth under temperature stress (cold, freezing and heat). Temperature stress cold germination experiments were carried out at 8° C. Heat stress germination experiments were conducted at 32° C. to 37° C. for 6 hours of exposure.

For Petri plate freeze tests, plants may be grown on Gamborg's B-5 medium without sucrose solidified with agar at 22° C. at about 100 μmol m⁻² s⁻¹ for 10 days. The plates are placed in a chamber at −2° C. for 2 hr, then ice nucleated. The plates are left in the dark for approximately 22 hr at −2° C., 24 hr at −5° C. and 24 hr at 4° C. The plates are then transferred to 22° C. in 24-hour light and scored three days later for survival. Whole plant freezing tests and electrolyte leakage freeze tests are performed as described (Haake et al. (2002) Plant Physiol. 130: 639-48; Gilmour et al. (2000) Plant Physiol. 124: 1854-1865)).

For nitrogen utilization assays, sterile seeds were sown onto plates containing media based on 80% MS without a nitrogen source (“low N germ” assay). For carbon/nitrogen balance (C/N) sensing assays, the media also contained 3% sucrose (−N/+G). The-“low N w/gIn germ” media was identical but was supplemented with 1 mM glutamine. Plates were incubated in a 24-hour light C (120-130 μEins⁻² in⁻¹) growth chamber at 22° C. Evaluation of germination and seedling vigor was done five days after planting for C/N assays. The production of less anthocyanin on these media is generally associated with increased tolerance to nitrogen limitation, and a transgene responsible for the altered response is likely involved in the plants ability to perceive their carbon and nitrogen status.

The transcription factor sequences of the present Sequence Listing, Tables, Figures, and their equivalogs can be used to prepare transgenic plants and plants with increased abiotic stress tolerance. The specific transgenic plants listed below are produced from sequences of the Sequence Listing, as noted. The Sequence Listing and Table 2 provide exemplary polynucleotide and polypeptide sequences of the invention.

### Example IX

### Mutagenesis of Plants Overexpressing an AP2 Transcription Factor

Transgenic plants overexpressing an AP2 polypeptide that confers stress tolerance may also be mutagenized to produce point or larger mutations, after which the plants may be screened for stress tolerance and desirable morphological characteristics.

Random mutagenesis is generally performed by methods well-known in the art (e.g. in Current Protocols in Molecular Biology, Ausubel et al. eds., Current Protocols, a joint venture between Greene Publishing Associates, Inc. and John Wiley & Sons, Inc., supplemented through 2000). Seeds or other plant material may be treated with a mutagenic chemical substance including, for example, diepoxybutane, diethyl sulfate, ethylene imine, ethyl methanesulfonate and N-nitroso-N-ethylurea. Alternatively, ionizing radiation from sources such as, for example, X-rays, gamma rays or fast neutron bombardment can be used. The seed or tissue materials are then generated into plants, and seed may be harvested from the plants.

### Example X

### Site Specific Mutagenesis

Once an advantageous mutation that causes a plant to retain normal or near-normal stature has been identified, it would be advantageous to confer this same mutation to other plants, including, for example, food crop or forestry species. This may be accomplished by incorporating and ectopically expressing a mutated transcription factor gene shown to confer abiotic or biotic stress tolerance without appreciable size reduction into a target plant, or by site specific mutation.

Site-specific mutagenesis uses oligonucleotide sequences that encode the DNA sequence with the desired mutation, in this case, a transcription factor sequence, as well as a sufficient number of adjacent nucleotides, to provide a primer sequence of sufficient size to form a stable duplex on both sides of the deletion junction being traversed. Typically, a primer of about 17 to about 75 nucleotides or more in length is preferred, with about 10 to about 25 or more residues on both sides of the junction of the sequence being altered.

Site-specific mutagenesis procedures are well known, and may be performed using a phage vector such as M13. This phage exists in both a single stranded and double stranded form. Alternatively, one may use a double-stranded plasmid rather than a phage, which would eliminate the recombinant method steps involved in transferring the gene of interest from a plasmid to a phage.

The next step is to prepare a vector using recombinant methods that contains the DNA sequence of interest encoding the subject transcription factor. A single-stranded vector may be used, or obtained by melting the two strands of a double stranded vector. An oligonucleotide primer that harbors the desired mutated sequence is then prepared, for example, by synthetic means, or using recombinant methods. This primer is then annealed with the single-stranded vector, followed by a treatment with a DNA polymerizing enzyme (e.g., polymerase 1 from E. coli, Klenow fragment). The DNA-polymerizing enzyme treatment causes a heteroduplex to be completed where one strand encodes the non-mutated sequence, and the second strand harbors the sequence containing the mutation. Cells of a plant species of interest are then transformed with the vector. This heteroduplex vector is then used to transform or transfect cells, and cells are selected that include recombinant vectors bearing the mutated sequence arrangement.

Alternatively, a gene amplification method (e.g., PCR) using, e.g., Taq polymerase, may be used to incorporate an oligonucleotide primer harboring the mutation of interest into an amplified DNA fragment that can then be cloned into an appropriate expression vector. A gene amplification method that makes use of a thermostable ligase and a thermostable polymerase may also be used to incorporate a phosphorylated mutagenic oligonucleotide into an amplified DNA fragment, that may then be cloned into an appropriate cloning or expression vector used to transform plant cells. For a further description of these methods and references, see, e.g., U.S. Pat. No. 6,635,806 or U.S. Pat. No. 6,620,988, from which the above site-specific mutagenesis methods were derived, Wu (ed.) Methods Enzymol. (1993) vol. 217, Academic Press, or Das et al (1995) Plant Cell. 7:287-294.

### Example XI

### Modification of non-CBF AP2 Transcription Factors

Plants overexpressing AP2 polypeptides that confer increased stress tolerance often grew at a reduced rate, were smaller, and may have experienced delayed flowering with respect to wild-type plants. These AP2 polypeptides include abiotic stress-conferring non-CBF sequences including G47 (SEQ ID NO: 10; e.g., in US patent publication no. US20030226173 and patent publication WO04031349) and their orthologs, paralogs and G867 (SEQ ID NO: 8; e.g., in US patent publication no. US20040098764) and its orthologs.

AP2 polypeptides that have also conferred abiotic stress tolerance when overexpressed in Arabidopsis plants includes G1792 (SEQ ID NO: 4; Table 1); for low nitrogen tolerance conferred by G1792, see US patent publication no. US20040098764; and for drought tolerance, see patent publication WO04031349).

These AP2 polypeptides may also include biotic stress (disease-tolerance)-conferring non-CBF sequences such as G1792 and its orthologs (see US patent publication no. US20040098764 and patent publication WO04031349), and G28 (AtERF1; SEQ ID NO: 6; encoded by GenBank accession number AB008103; Fujimoto et al. (2000) Plant Cell 12: 393-404; also see U.S. Pat. No. 6,664,446).

This Example provides a method for modifying non-CBF AP2/ERF or AP2/EREBP transcription factor polypeptides that comprise acidic residues in a region of the conserved AP2 domain corresponding to the ETAED (residues 179-183) of G28, AtERF1. Polypeptide variants are produced that, when overexpressed in plants, confer improved growth characteristics in comparison with the native AP2/ERF TF protein while retaining the desired trait conferred by transcription factor overexpression.

One embodiment of the invention is the modification of the residue corresponding to the glutamate residue 182 in G28 (ETAED) to one with lower acidity, such as a basic residue (e.g., lysine), or an aliphatic residue (e.g., alanine). The position corresponding to the glutamate residue 182 in G28 comprises either a glutamate residue in AP2/ERF polypeptides including G1792 (ETAEE), G47 (STAEG), or an aspartate residue in AP2/EREBP polypeptides such as G867 (NEEDE).

Other changes of single acidic residues in the motif comprise additional variants of the method. For those AP2/ERF proteins such as AtERF1 that contain multiple acidic residues, modification of more than one and optionally all acidic residues to basic or neutral residues is another embodiment. For example, for an AP2 sequence that contains a subsequence ETPAE corresponding to positions 179-183 in G28 (SEQ ID NO: 6); either or both of the two glutamate residues may be substituted by basic or neutral residues with the result that adverse morphological or developmental characteristics are reduced when this variant is overexpressed. Similarly, residues in a motif with three acidic residues, ESDVD; may be substituted with neutral or basic residues for the glutamate and/or aspartate residues, which may result in reduced adverse morphological or developmental characteristics when these variants are overexpressed. The same may be true for variants of, for example, G867 (SEQ ID NO: 8), which has four acidic residues in its corresponding motif (NEEDE, respectively).

The protein variants would be produced from a genetic construct in planta using variant genes developed through various, well-known methods in the art for site-specific mutation of a DNA sequence, such as the methods described in Example X. This Example also relates to plants transformed with such variants that retain the desired stress tolerance with diminished growth defects.

The analysis underlying this Example stems from the discovery of three variants that appear to eliminate secondary growth defects of CBF2 while retaining freezing tolerance in plants overexpressing these CBF2 variants. One of the mutations, a glutamate to lysine residue substitution, occurred in a region of the AP2/ERF protein that is highly conserved (the other two residues mutated in CBF2 do not correspond to conserved residues). By inspection of a complete alignment of AP2/ERF conserved domains, it became apparent that the glutamate to lysine substitution is in a loop (henceforth referred to as “the loop structure”) and the beginning of an α-helix of the conserved domain (based on AtERF1 structure, 1GCC.pdb in published protein databases). The additional acidic residues in some AP2 transcription factors suggest that the acidic amino acids likely play a role in interacting with a factor in transcriptional machinery. As noted above, modification of more than one and optionally all acidic residues to basic or aliphatic residues in this loop structure reduces adverse morphological or developmental characteristics in plants overexpressing AP2 transcription factor polypeptides comprising this loop structure.

Table 1 lists a number of AP2 sequences, many of which confer a stress-tolerant phenotype when a particular sequence has been overexpressed in Arabidopsis plants, as indicated in the last column identifying the general stress tolerance conferred. Each of these sequences contains a loop structure, relative to positions 179-183 in G28, comprising at least one acidic amino acid residue. CBF sequences tend to have one acidic residue at the fourth position, The majority of the non-CBF motifs have an acidic residue in the fourth position of their loop structures, and in many cases more than one acidic residue in their loop structures.

TABLE 1
AP2 sequences and acidic subsequences

| | | Species from which AP2 | | Type of Stress |
|---|---|---|---|---|
| SEQ ID | | Transcription Factor is | Loop | Tolerance |
| NO: | GID | Derived | Structure | Demonstrated |
| 2 | G912 | Arabidopsis thaliana | PTVEM | Abiotic |
| 4 | G1792 | Arabidopsis thaliana | ETAEE | Abiotic, biotic |
| 6 | G28 | Arabidopsis thaliana | ETAED | Biotic |
| 8 | G867 | Arabidopsis thaliana | NEEDE | Abiotic |
| 10 | G47 | Arabidopsis thaliana | STAEG | Abiotic |

The acidic amino acid residues in the loop structures found in Table 1, or similar structures found in other AP2 transcription factor polypeptides, may be substituted with basic or neutral amino acid residues. When overexpressed in plants, these mutated AP2 transcription factor polypeptides may also confer stress tolerance, but with fewer or reduced adverse morphological characteristics such as decreased seed production, reduced size, increased size, reduced fertility, and delayed flowering.

### Example XII

### Transformation of Non-Arabidopsis Species

For monocot plants, a vector comprising the modified sequence may be introduced into monocot plants by well known means, including direct DNA transfer or Agrobacterium tunefaciens-mediated transformation.

It is routine to produce transgenic plants using most dicot plants (e.g., in Weissbach and Weissbach, (1989) supra, Gelvin et al. (1990) supra, Herrera-Estrella et al. (1983) supra, Bevan (1984) supra, and Klee (1985) supra). For example, numerous protocols for the transformation of tomato and soy plants have been previously described, and are well known in the art. Gruber et al. ((1993) in Methods in Plant Molecular Biology and Biotechnology, p. 89-119, Glick and Thompson, eds., CRC Press, Inc., Boca Raton) describe several expression vectors and culture methods that may be used for cell or tissue transformation and subsequent regeneration.

There are a substantial number of alternatives to Agrobacterium-mediated transformation protocols for transferring exogenous genes into soybeans or tomatoes. For soybean transformation, methods are described by Mild et al. (1993) in Methods in Plant Molecular Biology and Biotechnology, p. 67-88, Glick and Thompson, eds., CRC Press, Inc., Boca Raton; and U.S. Pat. No. 5,563,055, (Townsend and Thomas), issued Oct. 8, 1996. One such method is microprojectile-mediated transformation, in which DNA on the surface of microprojectile particles is driven into plant tissues with a biolistic device (e.g., in Sanford et al., (1987) Part. Sci. Technol. 5:27-37; Christou et al. (1992) Plant. J. 2: 275-281; Sanford (1993) Methods Enzymol. 217: 483-509; Klein et al. (1987) Nature 327: 70-73; U.S. Pat. No. 5,015,580 (Christou et al), issued May 14, 1991; and U.S. Pat. No. 5,322,783 (Tomes et al.), issued Jun. 21, 1994).

Alternatively, sonication methods (for example, in Zhang et al. (1991) Bio/Technology 9: 996-997); direct uptake of DNA into protoplasts using CaCl₂ precipitation, polyvinyl alcohol or poly-L-ornithine (for example, in Hain et al. (1985) Mol. Gen. Genet. 199: 161-168; Draper et al., Plant Cell Physiol. 23: 451-458 (1982)); liposome or spheroplast fusion (for example, in Deshayes et al. (1985) EMBO J., 4: 2731-2737; Christou et al. (1987) Proc. Natl. Acad. Sci. U.S.A. 84: 3962-3966); and electroporation of protoplasts and whole cells and tissues (for example, in Donn et al. (1990) in Abstracts of VIIth International Congress on Plant Cell and Tissue Culture IAPTC, A2-38: 53; D'Halluin et al. (1992) Plant Cell 4: 1495-1505; and Spencer et al. (1994) Plant Mol. Biol. 24: 51-61) have been used to introduce foreign DNA and expression vectors into plants.

After plants or plant cells are transformed (and the latter regenerated into plants) the transgenic plant thus generated may be crossed with itself (“selfing”) or a plant from the same line, a non-transformed or wild-type plant, or another transformed plant from a different transgenic line of plants. Crossing provides the advantages of being able to produce new and perhaps stable transgenic varieties. Genes and the traits they confer that have been introduced into a tomato or soybean line may be moved into distinct line of plants using traditional backcrossing techniques well known in the art. Transformation of tomato plants may be conducted using the protocols of Koornneef et al (1986), in Tomato Biotechnology: Alan R. Liss, Inc., 169-178, and in U.S. Pat. No. 6,613,962, the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 μM α-naphthalene acetic acid and 4.4 μM 6-benzylaminopurine. The explants are then infected with a diluted overnight culture of Agrobacterium tumefaciens containing an expression vector comprising a polynucleotide of the invention for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (w/v/) sucrose, pH 5.7) to an OD₆₀₀ of 0.8.

Following the cocultivation, the cotyledon explants are transferred to Petri dishes with selective medium consisting of MS medium supplemented with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a medium containing kanamycin sulfate is regarded as a positive indication of a successful transformation.

Transformation of soybean plants may be conducted using the methods found in, for example, U.S. Pat. No. 5,563,055 (Townsend et al., issued Oct. 8, 1996), described in brief here. In this method soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the expression vector comprising a polynucleotide of the invention are grown to log phase, pooled, and concentrated by centrifugation. Inoculations are conducted in batches such that each plate of seed was treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium which has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (e.g., U.S. Pat. No. 5,563,055).

The explants may then be picked, embedded and cultured in solidified selection medium. After one month on selective media transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

The polynucleotide and polypeptide sequences derived from monocots may be used to transform both monocot and dicot plants, and those derived from dicots may be used to transform either group, although some of these sequences will function best if the gene is transformed into a plant from the same group as that from which the sequence is derived.

Transformed plants that are abiotic or biotic stress-tolerant may then be identified by, for example, subjecting seeds of these transformed plants to abiotic stress assays, including germination assays (e.g., a high sucrose germination assay to measure sucrose sensing) or pathogen challenge (e.g., Fusarium). Sterile monocot seeds, including, but not limited to, corn, rice, wheat, rye and sorghum, as well as dicots including, but not limited to soybean and alfalfa, are sown on 80% MS medium plus vitamins with 9.4% sucrose; control media lack sucrose. All assay plates are then incubated at 22° C. under 24-hour light, 120-130 μEin/m²/s, in a growth chamber. Evaluation of germination and seedling vigor is then conducted three days after planting. Overexpressors of these genes may be found to be more tolerant to high sucrose by having better germination, longer radicles, and more cotyledon expansion. These results would indicate that overexpressors of mutant AP2 transcription factors are involved in sucrose-specific sugar sensing.

Plants overexpressing these variants may also be subjected to soil-based drought assays to identify those lines that are more tolerant to water deprivation than wild-type control plants. Generally, plants that overexpress a AP2 mutant or variant polypeptide will appear significantly larger and greener, with less tissue damage, wilting, desiccation, or necrosis, than wild-type controls plants, particularly after a period of freezing or water deprivation. Abiotic or biotic stress-tolerant plants that are morphologically and developmentally similar to wild-type plants may then be used to generate lines for commercial development.

### Example XIII

### Mutagenesis of non-Arabidopsis Sequences Encoding AP2 Transcription Factors

Similar to the methods described in the above Examples, seeds overexpressing an AP2 polypeptide derived from any of a number of diverse plant species are mutagenized (e.g., with EMS as described above and in Somerville and Ogren (1982) in Edelman, Hallick, and Chua, eds, Methods in Chloroplast Molecular Biology. Elsevier Biomedical Press, Amsterdam, The Netherlands, pp 129-138). The AP2 sequences or the seeds can be derived from any plant including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf; or fruits or fruit trees, vegetables such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and brassicas (such as broccoli, cabbage, cauliflower, Brussels sprouts, and kohlrabi). Seeds of other crops, including fruits and vegetables, whose phenotype can be changed and which comprise homologous AP2 sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassaya, turnip, radish, yam, and sweet potato; and beans. The AP2 sequences or seeds may also be derived from woody species, such pine, poplar and eucalyptus, or mint or other labiates. In addition, seeds or AP2 sequences may be derived from plants that are evolutionarily related to crop plants, but which may not have yet been used as crop plants. Examples include deadly nightshade (Atropa belladona), related to tomato; jimson weed (Datura strommium), related to peyote; and teosinte (Zea species), related to corn (maize).

After mutagenesis, the seeds are planted out into separate flats of soil and grown. The seed is collected individually from each flat and M2 plants are screened for those that are phenotypically more similar to wild type than plants constitutively overexpressing a homologous AP2 polypeptide. M3 seed is collected from individual putative mutants and retained for further study. Where populations of M3 plants from an individual M2 plant are not uniform in size, M4 or M5 seeds are collected from individual M3 or M4 plants.

In addition to the above method that may be used to generate a random mutation, site specific mutagenesis may be used to produce mutations in a AP2 gene that is orthologous to a polynucleotide sequence that is shown to function in this manner, using, for example, the methods described in the previous example.

Analysis of the expression profile of transcription factor mutants generated using these methods or others may be performed using a variety of methods, for example, by Northern analysis or RT-PCR, to determine the level of gene expression of gene products that may be associated with abiotic or biotic stress tolerance. For example, the expression of COR genes by G912 transgene mutants would confirm that the reason for regaining normal or near normal stature was not due to a loss of transcription factor RNA expression but rather an alteration in the encoded sequence of the mRNA.

For phenotypic analysis, seeds from plants transformed with mutant or variant polypeptides are grown and the plants are screened for those that more closely resembled wild-type plants in growth and development.

### Example XIV

### Results from Variant Studies

The results of the studies of plants overexpressing truncated or GAL4 fusion versions of the transcription factor proteins of the invention clearly indicated that variations of these transcription factor can be generated that confer stress tolerance in plants and/or eliminate or greatly reducing the adverse morphological effects that generally result from overexpression of the native protein.

For example, overexpression of P21197 (SEQ ID NO: 18), which comprised a GAL4 transactivation domain fused to the N terminus of the G912 protein, produced a striking phenotype. Lines overexpressing this construct were dark in coloration, late flowering (up to 3-4 weeks after wild type in 24-hour light conditions) and exhibited greater rosette biomass than wild type at later stages of development. This phenotype was highly penetrant being observed in 15 out of 16 T1 lines (all lines from batch 1261-1276 except #1261). Lines #1263, 1264, 1266, 1267, 1269, 1272, 1275, 1276. A significant number of lines of these plants were shown to be significantly more cold and freezing tolerant than wild-type control plants.

Overexpression of P21194 (SEQ ID NO: 17), encoding a G912 clone that had a GAL4 transactivation domain fused at the C terminus of the G912 polypeptide (35S::G912-GAL4), resulted in plants that displayed some variation in size, but no consistent differences in morphology to controls. A significant number of the lines overexpressing P21194 were shown to be more salt, ABA, and sucrose tolerant than wild-type control plants.

Two batches of T1 plants overexpressing P21270 (SEQ ID NO: 16), an overexpression construct encoding a truncated version of the G912 protein comprising only the AP2 domain and the two CBF boxes, showed any consistent differences in morphology to controls. A significant number of the lines overexpressing P21270 were shown to be more salt, drought, and mannitol tolerant than wild-type control plants.

Plants constitutively overexpressing G47 (SEQ ID NO: 9; for example, 35S::G47) often are tolerant to a number of hyperosmotic and drought related stresses, but also show a high frequency of dwarfing, retarded growth rates, and morphological abnormalities. However, overexpression of P25186 (SEQ ID NO: 40), which comprised a GAL4 transactivation domain fused to the N terminus of the G47 protein (35S::GAL4-G47), produced plants that appeared very vigorous, exhibited early flowering and maturation and did not show the high frequency of dwarfing, retarded growth rate, and morphological abnormalities that were prevalent in 35S::G47 lines. Surprisingly, the ³⁵S::GAL4-G47 lines appeared more vigorous than wild type in a number of lines.

Table 2 lists the constructs of the invention that demonstrated that a number of approaches, that is, truncations, deletions, point mutations and protein fusions, may be used to mutate AP2 transcription factors and greatly to limit, or prevent, deleterious effects resulting from overexpression of these sequences. In some cases, the stress-tolerant phenotype was not as strong as that observed in transgenic plants constitutively overexpressing the AP2 sequences. In other cases, the stress-tolerant phenotype was similar to that conferred by constitutive overexpression. Control plants included wild type or plants transformed with the target construct vector lacking the transcription factor gene.

TABLE 2
Constructs used to produce transgenic plants with abiotic and/or biotic stress tolerance
with reduced adverse morphological effects relative to control plants.

| | Construct | | | |
|---|---|---|---|---|
| | SEQ ID | Construct | Increase | |
| Construct | No. | encodes: | tolerance/resistance | Plant morphology and/or size |
| P174 | 13 | G28 | Scl Ery Bot Ery | Small relative to controls |
| P25678 | 26 | G28 point | Scl Ery | Two lines were slightly late in development; |
| | | mutation #1 | | most lines were similar in size and morphology |
| | | | | to controls |
| P25680 | 27 | G28 point | Scl Ery | Lines occasionally had curling leaves, slightly |
| | | mutation #3 | | early in development; most lines were similar |
| | | | | in size and morphology to controls |
| P25682 | 28 | G28 point | Scl Ery | Lines were occasionally slightly early in |
| | | mutation #5 | | development, a few lines had broad leaves, |
| | | | | most lines were similar in size and morphology |
| | | | | to controls |
| P25684 | 29 | G28 point | Ery | Occasionally slightly late in development, |
| | | mutation #7 | | most lines were similar in size and |
| | | | | morphologically similar to controls |
| P21143 | 30 | G28 C-GAL | Scl Ery | A few lines were small and dark green; most |
| | | fusion | | others were similar in size and morphology to |
| | | | | controls |
| P21196 | 31 | G28 N-GAL | Scl | Some size variation but lines were generally |
| | | fusion | | were similar in size and morphology to |
| | | | | controls |
| P894 | 15 | G47 | NaCl drt | Lines generally small with contorted, curling |
| | | | | leaves and delayed flowering; a few lines were |
| | | | | larger than controls late in their development |
| P25732 | 37 | G47 point | cold des | A few lines were slightly late developing, but |
| | | mutation #1 | | most lines were similar in size and morphology |
| | | | | to controls |
| P25733 | 38 | G47 point | des | Upright, curling, twisting leaves, late |
| | | mutation #2 | | developing, generally smaller than controls but |
| | | | | a few lines had larger rosettes than controls |
| P25735 | 39 | G47 point | cold des | Late developing, some lines were smaller early |
| | | mutation #4 | | in development, similar in morphology and |
| | | | | size or larger than controls later in |
| | | | | development |
| P25186 | 40 | G47 N-GAL | man des | Possibly slightly early developing, but were |
| | | fusion | | generally were similar in size and morphology |
| | | | | to controls |
| P25279 | 41 | G47 GFP | des drt | Bushy rosettes with twisted leaves, smaller in |
| | | fusion | | size, especially early in development, some |
| | | | | lines were similar in size to controls late in |
| | | | | development |
| P383 or | 14 or 42 | G867 | NaCl suc ABA | Small size relative to controls |
| P7140 | | | cold drt | |
| P21276 | 32 | G867 | heat cold | A few lines were small, others had more |
| | | dominant | | rosette leaves than controls, most lines were |
| | | negative | | similar in size and morphology to controls |
| | | deletion in | | |
| | | secondary | | |
| | | domain | | |
| P21275 | 33 | G867 | heat cold des | Slightly early, a few lines were small, others |
| | | dominant | | were morphological similar and similar in size |
| | | negative | | or larger relative to controls |
| | | deletion | | |
| P21193 | 34 | G867 C-GAL | suc cold | Some size variation, a few lines were small, |
| | | fusion | | some lines were similar in size and |
| | | | | morphology to controls |
| P21201 | 35 | G867 N-GAL | NaCl drt | A few lines slightly late flowering, dark green, |
| | | fusion | | otherwise morphologically similar to controls |
| P25301 | 36 | G867 GFP | ABA drt | Narrow, upright leaves, late developing vs. |
| | | fusion | | controls; some lines were smaller, others were |
| | | | | similar in size and morphology to controls |
| P393 or | 11 or 43 | G912 | glu frz drt | G912 overexpressors were tiny to small, dark |
| P3366 | | | | green, delayed flowering relative to controls |
| P21270 | 16 | G912 | des | Similar in size and morphology to controls |
| | | dominant | | |
| | | negative | | |
| | | deletion | | |
| P21194 | 17 | G912 C-GAL | NaCl suc ABA | Some size variation, but generally were similar |
| | | fusion | | in size and morphology to controls |
| P21197 | 18 | G912 N-GAL | cold frz drt | Slightly dark in coloration, delay in flowering, |
| | | fusion | | were similar in size or larger than controls |
| P1695 | 12 | G1792 | cold des N drt Bot | G1702 overexpressors were generally small |
| | | | Fus Ery | relative to controls |
| P25437 | 19 | G1792 | suc | Lines occasional possessed a large rosette size |
| | | dominant | | with long leaves, wer slightly early flowering, |
| | | negative | | but otherwise were similar in size and |
| | | deletion | | morphology to controls |
| P25739 | 20 | G1792 point | cold suc des drt N | Dull green, flat, serrated leaves, ranging from |
| | | mutation #2 | Ery | small, especially at seedling stage, to similar in |
| | | | | size as controls |
| P25740 | 21 | G1792 point | suc N | Dull green, flat, serrated leaves, ranging from |
| | | mutation #3 | | small to similar in size as controls |
| P25741 | 22 | G1792 point | suc des cold N | Slightly late developing, bushy, curling leaves, |
| | | mutation #4 | | some lines were small, some lines were larger |
| | | | | with larger rosettes than controls |
| P25083 | 23 | G1792 C- | N | Lines were late developing, darker in |
| | | GAL fusion | | coloration, shiny, and ranged in size from |
| | | | | similar in size to larger than controls |
| P25093 | 24 | G1792 N- | cold des N Ery | Lines were late developing, darker in |
| | | GAL fusion | | coloration, shiny, had upward pointing leaves, |
| | | | | sized ranged from slightly smaller to larger |
| | | | | than controls |
| P25271 | 25 | G1792 GFP | cold des drt N | Dark green, shiny, with size that ranged from |
| | | fusion | | slightly small to similar in size as controls |

Abbreviations used in Table 2:
ABA - less sensitive to abscisic acid than controls
Bot - greater resistance to Botrytis than controls
des - more tolerant than controls in plate based desiccation assay
drt - more tolerant than controls in soil-based drought assay
Ery - greater resistance to Erysiphe than controls
frz - more tolerant than controls in freezing assay
Fus - greater resistance to Fusarium than controls
GFP - green fluorescent protein
glu - more tolerant than controls in glucose assay
N - more tolerant than controls in low nitrogen tolerance
NaCl - more tolerant than controls in salt assay
Scl - greater resistance to Sclerotinia than controls
suc - more tolerant than controls in sucrose assay

### Example XV

### Application of Altered Arabidopsis and Non-Arabidopsis AP2 Transcription Factors in Plants

A sizeable number of AP2 polypeptides derived from diverse species have been shown to confer tolerance to a number of abiotic stresses (e.g., desiccation, drought, hyperosmotic stress, low nutrient stress, high salt, heat and cold), and include orthologs from Glycine max, Oryza sativa, Zea mays, Medicago sativa, and Medicago truncatula of G912 (CBF4; SEQ ID NO: 2), G1792 (SEQ ID NO: 4), G28 (SEQ ID NO: 6), G867 (SEQ ID NO: 8), and G47 (SEQ ID NO: 10). Similarly, soy and rice orthologs of G1792 (SEQ ID NO: 4) and G28 (SEQ ID NO: 6) have been shown to confer biotic stress tolerance, including fungal disease tolerance, in plants when overexpressed. Once any sequence of the invention, that is a modified or mutated AP2 transcription factor including SEQ ID Nos. 2, 4, 6, 8, 10, or their paralogs or orthologs, has been overexpressed in plants, these plants may be selected on the basis of greater tolerance to an environmental stress than a wild-type or other control plant grown for the same length of time. These stresses may include drought, desiccation, salt, freezing, heat, cold, Erysiphe infection, Botrytis infection, Sclerotinia infection, and Fusarium infection. These plants may also be selected on the basis of reduced adverse or developmental morphological characteristics relative to plants transformed with the same AP2 transcription factor, but lacking the mutation. These reduced verse or developmental morphological characteristics may include decreased seed production, reduced size, increased size, reduced fertility, or delayed flowering, among others.

Modifications (mutations, truncations, fusions) similar to those described in the above Examples for Arabidopsis AP2 sequences are expected to perform similarly and confer abiotic or biotic stress tolerance when overexpressed with few adverse morphological or developmental effects. These sequences, including protein fusion, truncated sequences, and mutant forms of the AP2 sequences, and transgenic plants generated with these protein variants, are thus encompassed by the present invention. Methods for conferring stress tolerance in plants of wild-type or nearly wild-type morphology and fertility are also encompassed by the present invention.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

1. A first transgenic plant comprising an expression vector comprising a first recombinant polynucleotide, wherein:
the recombinant polynucleotide encodes an AP2 transcription factor that is mutated;
the transgenic plant is larger than a second transgenic plant comprising a second recombinant polynucleotide that encodes the AP2 transcription factor that has not been mutated; and
the first transgenic plant is more tolerant to an abiotic stress or more resistant to a disease pathogen than a wild-type plant of the same species.

2. The transgenic plant of claim 1, wherein the transgenic plant is similar in size to a wild-type plant of the same species grown for the same length of time.

3. The transgenic plant of claim 1, wherein the transgenic plant is larger in size than a wild-type plant of the same species grown for the same length of time.

4. The transgenic plant of claim 1, wherein the transgenic plant is morphologically similar to the wild-type plant.

5. The transgenic plant of claim 1, wherein the AP2 transcription factor that is mutated comprises a point mutation, a deletion, a truncation, or a protein fusion, as compared to the AP2 transcription factor that has not been mutated.

6. The transgenic plant of claim 1, wherein the transgenic plant comprises SEQ ID NO: 18.

7. The transgenic plant of claim 1, wherein the abiotic stress is selected from the group consisting of low nitrogen conditions, drought, desiccation, salt, freezing, heat, and cold.

8. The transgenic plant of claim 1, wherein the disease pathogen is selected from the group consisting of Erysiphe, Botrytis, Sclerotinia and Fusarium.

9. Use of an expression vector comprising a first recombinant polynucleotide that encodes an AP2 transcription factor that is mutated to produce a first transgenic plant that is more stress tolerant and larger than a second transgenic plant comprising a second recombinant polynucleotide that encodes the AP2 transcription factor that has not been mutated, and the stress is selected from the group consisting of low nitrogen conditions, drought, desiccation, salt, freezing, heat, cold, Erysiphe infection, Botrytis infection, Sclerotinia infection, and Fusarium infection.

10. The use of claim 9, wherein the first transgenic plant is larger in size than a wild-type plant of the same species grown for the same length of time.

11. A method for producing a first transgenic plant that is larger than a second transgenic plant and more stress tolerant than a wild-type plant of the same species, and the stress is selected from the group consisting of low nitrogen conditions, drought, desiccation, salt, freezing, heat, cold, Erysiphe infection, Botrytis infection, Sclerotinia infection, and Fusarium infection, the methods steps including:
transforming a first plant with a first expression vector that encodes an AP2 transcription factor that is mutated to produce the first transgenic plant;
transforming a second plant with a second expression vector that encodes the AP2 transcription factor that is not mutated to produce the second transgenic plant; and
selecting the first transgenic plant on the basis of larger size than the second transgenic plant.

12. The method of claim 11, wherein the first transgenic plant is similar in size to the wild-type plant when the first transgenic plant and the wild-type plant are grown for the same length of time.

13. The method of claim 11, wherein the first transgenic plant comprises SEQ ID NO: 18 and the second transgenic plant comprises SEQ ID NO: 11.

14. A method for increasing tolerance to an environmental stress and reducing adverse or developmental morphological characteristics in a plant, when the stress is selected from the group consisting of low nitrogen conditions, drought, desiccation, salt, freezing, heat, cold, Erysiphe infection, Botrytis infection, Sclerotinia infection, and Fusarium infection, and the adverse or developmental morphological characteristics are selected from the group consisting of decreased seed production, reduced size, increased size, reduced fertility, and delayed flowering, relative to a wild-type plant of the same species grown for the same length of time, the methods steps including:
transforming a first plant with a first expression vector that encodes an AP2 transcription factor that is mutated to produce a first transgenic plant;
transforming a second plant with a second expression vector that encodes the AP2 transcription factor that is not mutated to produce a second transgenic plant; and
selecting the first transgenic plant on the basis of larger size than the second transgenic plant and greater
tolerance or resistance to the environmental stress than the wild-type plant of the same species as the first transgenic plant.

15. The transgenic plant of claim 2, wherein the transgenic plant is morphologically similar to the wild-type plant.


SEQUENCE LISTING (44 sequences)

SEQ ID NO: 1 - DNA, 724 bp, Arabidopsis thaliana
  G912
catcttatccaaagaaaaaatgaatccattttactctacattcccagactcgtttctctcaatctccgatcatagatctccggtttcagacagtagtgagtgttcaccaaagttagcttcaagttgtccaaagaaacgagctgggaggaagaagtttcgtgagacacgtcatccgatttacagaggagttcgtcagaggaattctggtaaatgggtttgtgaagttagagagcctaataagaaatctaggatttggttaggtacttttccgacggttgaaatggctgctcgtgctcatgatgttgctgctttagctcttcgtggtcgctctgcttgtctcaatttcgctgattctgcttggcggcttcgtattcctgagactacttgtcctaaggagattcagaaagctgcgtctgaagctgcaatggcgtttcagaatgagactacgacggagggatctaaaactgcggcggaggcagaggaggcggcaggggagggggtgagggagggggagaggagggcggaggagcagaatggtggtgtgttttatatggatgatgaggcgcttttggggatgcccaacttttttgagaatatggcggaggggatgcttttgccgccgccggaagttggctggaatcataacgactttgacggagtgggtgacgtgtcactctggagttttgacgagtaattttttggctctttttctggataataagtt

SEQ ID NO: 2 - protein, 224 aa, Arabidopsis thaliana
  G912 polypeptide_conserved domain in aa coordinates 51-118
MNPFYSTFPDSFLSISDHRSPVSDSSECSPKLASSCPKKRAGRKKFRETRHPIYRGVRQRNSGKWVCEVREPNKKSRIWLGTFPTVEMAARAHDVAALALRGRSACLNFADSAWRLRIPETTCPKEIQKAASEAAMAFQNETTTEGSKTAAEAEEAAGEGVREGERRAEEQNGGVFYMDDEALLGMPNFFENMAEGMLLPPPEVGWNHNDFDGVGDVSLWSFDE

SEQ ID NO: 3 - DNA, 696 bp, Arabidopsis thaliana
  G1792
aatccatagatctcttattaaataacagtgctgaccaagctcttacaaagcaaaccaatctagaacaccaaagttaatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagataatcacgattagttttgttttgatattttatgtggcactgttgtggctacctacgtgcattatgtgcatgtataggtcgcttgattagtactttataacatgcatgccacgaccataaattgtaagagaagacgtactttgcgttttcatgaaatatgaatgttagatggtttgagtacaaaaaaaaaaaaaaaaaaaaaaa

SEQ ID NO: 4 - protein, 139 aa, Arabidopsis thaliana
  G1792 polypeptide_conserved domain in aa coordinates 16-80
MESSNRSSNNQSQDDKQARFRGVRRRPWGKFAAEIRDPSRNGARLWLGTFETAEEAARAYDRAAFNLRGHLAILNFPNEYYPRMDDYSLRPPYASSSSSSSSGSTSTNVSRQNQREVFEFEYLDDKVLEELLDSEERKR

SEQ ID NO: 5 - DNA, 964 bp, Arabidopsis thaliana
  G28
gaaatctcaacaagaaccaaaccaaacaacaaaaaaacattcttaataattatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattaaaaaaaaaaaaaaaaaa

SEQ ID NO: 6 - protein, 268 aa, Arabidopsis thaliana
  G28 polypeptide_conserved domain in aa coordinates 145-208
MSMTADSQSDYAFLESIRRHLLGESEPILSESTASSVTQSCVTGQSIKPVYGRNPSFSKLYPCFTESWGDLPLKENDSEDMLVYGILNDAFHGGWEPSSSSSDEDRSSFPSVKIETPESFAAVDSVPVKKEKTSPVSAAVTAAKGKHYRGVRQRPWGKFAAEIRDPAKNGARVWLGTFETAEDAALAYDRAAFRMRGSRALLNFPLRVNSGEPDPVRIKSKRSSFSSSNENGAPKKRRTVAAGGGMDKGLTVKCEVVEVARGDRLLVL

SEQ ID NO: 7 - DNA, 1281 bp, Arabidopsis thaliana
  G867
cacaacacaaacacatttctgttttctccattgtttcaaaccataaaaaaaaacacagattaaatggaatcgagtagcgttgatgagagtactacaagtacaggttccatctgtgaaaccccggcgataactccggcgaaaaagtcgtcggtaggtaacttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttcaccggagagttcaagaaacgacgtcgtaggaaacaaaagagtgaacgatactgagatgttatcgttggtgtgtagcaagaagcaacgcatctttcacgcctcgtaacaactcttcttctttttttttcttttgttgttttaataatttttaaaaactccattttcgttttctttatttgcatcggtttctttcttcttgtttaccaaaggttcatgagttgtttttgttgtattgatgaactgtaaattttatttataggataaattttaaaaaaaaaaaaaaaaaaaa

SEQ ID NO: 8 - protein, 344 aa, Arabidopsis thaliana
  G867 polypeptide_conserved domain in aa coordinates 59-124, 184-276
MESSSVDESTTSTGSICETPAITPAKKSSVGNLYRMGSGSSVVLDSENGVEAESRKLPSSKYKGVVPQPNGRWGAQIYEKHQRVWLGTFNEEDEAARAYDVAVHRFRRRDAVTNFKDVKMDEDEVDFLNSHSKSEIVDMLRKHTYNEELEQSKRRRNGNGNMTRTLLTSGLSNDGVSTTGFRSAEALFEKAVTPSDVGKLNRLVIPKHHAEKHFPLPSSNVSVKGVLLNFEDVNGKVWRFRYSYWNSSQSYVLTKGWSRFVKEKNLRAGDVVSFSRSNGQDQQLYIGWKSRSGSDLDAGRVLRLFGVNISPESSRNDVVGNKRVNDTEMLSLVCSKKQRIFHAS

SEQ ID NO: 9 - DNA, 785 bp, Arabidopsis thaliana
  G47
cttcttcttcacatcgatcatcatacaacaacaaaaaatggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccgtagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgatttatctcgacggtcatgatcacgtttgatcttcttttgagtaagattttgtaccataatcaaaacaggtgtggtgctaaaatcttactcaaaacaagattaggtaccacagagaaacaatcaaatggttgtgaatatacattataaggttttgattaatgtttgtttcactgatttagtgaagtttggtccattgtatacaaatctattcaagaaacctagcgcgagatcatgtttcgtgattgaagattgagatttttaagtattcgtaatatttttgtaaaatacaaataaaaaaaaaaaaaaaaaaaaa

SEQ ID NO: 10 - protein, 144 aa, Arabidopsis thaliana
  G47 polypeptide_conserved domain in aa coordinates 10-75
MDYRESTGESQSKYKGIRRRKWGKWVSEIRVPGTRDRLWLGSFSTAEGAAVAHDVAFFCLHQPDSLESLNFPHLLNPSLVSRTSPRSIQQAASNAGMAIDAGIVHSTSVNSGCGDTTTYYENGADQVEPLNISVYDYLGGHDHV

SEQ ID NO: 11 - DNA, 724 bp, artificial sequence
  P393 nucleic acid construct encoding G912 from A. thaliana
catcttatccaaagaaaaaatgaatccattttactctacattcccagactcgtttctctcaatctccgatcatagatctccggtttcagacagtagtgagtgttcaccaaagttagcttcaagttgtccaaagaaacgagctgggaggaagaagtttcgtgagacacgtcatccgatttacagaggagttcgtcagaggaattctggtaaatgggtttgtgaagttagagagcctaataagaaatctaggatttggttaggtacttttccgacggttgaaatggctgctcgtgctcatgatgttgctgctttagctcttcgtggtcgctctgcttgtctcaatttcgctggttctgcttggcggcttcgtattcctgagactacttgtcctaaggagattcagaaagctgcgtctgaagctgcaatggcgtttcagaatgagactacgacggagggatctaaaactgcggcggaggcagaggaggcggcaggggagggggtgagggagggggagaggagggcggaggagcagaatggtggtgtgttttatatggatgatgaggcgcttttggggatgcccaacttttttgagaatatggcggaggggatgcttttgccgccgccggaagttggctggaatcataacgactttgacggagtgggtgacgtgtcactctggagttttgacgagtaattttttggctctttttctggataataagtt

SEQ ID NO: 12 - DNA, 673 bp, artificial sequence
  P1695 nucleic acid construct encoding G1792 from A. thaliana
aatccatagatctcttattaaataacagtgctgaccaagctcttacaaagcaaaccaatctagaacaccaaagttaatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagataatcacgattagttttgttttgatattttatgtggcactgttgtggctacctacgtgcattatgtgcatgtataggtcgcttgattagtactttataacatgcatgccacgaccataaattgtaagagaagacgtactttgcgttttcatgaaatatgaatgttagatggtttgagtac

SEQ ID NO: 13 - DNA, 964 bp, artificial sequence; identical to SEQ ID NO: 5
  P174 nucleic acid construct encoding G28 from A. thaliana

SEQ ID NO: 14 - DNA, 1281 bp, artificial sequence; identical to SEQ ID NO: 7
  P383 nucleic acid construct encoding G867 from A. thaliana

SEQ ID NO: 15 - DNA, 505 bp, artificial sequence
  P894 nucleic acid construct encoding G47 from A. thaliana
cttcttcttcacatcgatcatcatacaacaacaaaaaatggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccgtagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgatttatctcgacggtcatgatcacgtttgatctt

SEQ ID NO: 16 - DNA, 315 bp, artificial sequence
  P21270 nucleic acid construct encoding a truncated form of G912 protein from A. thaliana comprising the AP2 domain and 2 CBF boxes(a dominant negative deletion)
tcgacgtgagtgttcaccaatgttagcttcaagttgtccaaagaaacgagctgggaggaagaagtttcgtgagacacgtcatccgatttacagaggagttcgtcagaggaattctggtaaatgggtttgtgaagttagagagcctaataagaaatctaggatttggttaggtacttttccgacggttgaaatggctgctcgtgctcatgatgttgctgctttagctcttcgtggtcgctctgcttgtctcaatttcgctgattctgcttggcggcttcgtattcctgagactacttgacctaaggagattcaggc

SEQ ID NO: 17 - DNA, 1045 bp, artificial sequence
  P21194 overexpression construct encoding a G912 clone from A. thaliana with a GAL4 transactivation domain fused at C-terminus(35S::G912-GAL4)
catcttatccaaagaaaaaatgaatccattttactctacattcccagactcgtttctctcaatctccgatcatagatctccggtttcagacagtagtgagtgttcaccaaagttagcttcaagttgtccaaagaaacgagctgggaggaagaagtttcgtgagacacgtcatccgatttacagaggagttcgtcagaggaattctggtaaatgggtttgtgaagttagagagcctaataagaaatctaggatttggttaggtacttttccgacggttgaaatggctgctcgtgctcatgatgttgctgctttagctcttcgtggtcgctctgcttgtctcaatttcgctgattctgcttggcggcttcgtattcctgagactacttgtcctaaggagattcagaaagctgcgtctgaagctgcaatggcgtttcagaatgagactacgacggagggatctaaaactgcggcggaggcagaggaggcggcaggagagggggtgagggagggggagaggagggcggaggagcagaatggtggtgtgttttatatggatgatgaggcgcttttggggatgcccaacttttttgagaatatggcggaggggatgcttttgccgccgccggaagttggctggaatcataacgactttgacggagtgggtgacgtgtcactctggagttttgacgagcaattccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagtaa

SEQ ID NO: 18 - DNA, 1059 bp, artificial sequence
  P21197 overexpression construct encoding a G912 clone from A.thaliana that has a GAL4 transactivation domain fused at the N terminus(35S::GAL4-G912)
atgcccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagggtaccgtcgacaatccattttactctacattcccagactcgtttctctcaatctccgatcatagatctccggtttcagacagtagtgagtgttcaccaaagttagcttcaagttgtccaaagaaacgagctgggaggaagaagtttcgtgagacacgtcatccgatttacagaggagttcgtcagaggaattctggtaaatgggtttgtgaagttagagagcctaataagaaatctaggatttggttaggtacttttccgacggttgaaatggctgctcgtgctcatgatgttgctgctttagctcttcgtggtcgctctgcttgtctcaatttcgctgattctgcttggcggcttcgtattcctgagactacttgtcctaaggagattcagaaagctgcgtctgaagctgcaatggcgtttcagaatgagactacgacggagggatctaaaactgcggcggaggcagaggaggcggcaggggagggggtgagggagggggagaggagggcggaggagcagaatggtggtgtgttttatatggatgatgaggcgcttttggggatgcccaacttttttgagaatatggcggaggggatgcttttgccgccgccggaagttggctggaatcataacgactttgacggagtgggtgacgtgtcactctggagttttgacgagtaattttttggctctttttctggataataagtt

SEQ ID NO: 19 - DNA, 348 bp, artificial sequence
  P25437  overexpression construct carrying a truncated form of G1792 from A. thaliana that comprises only the first 115 amino acids35S::G1792(aa1-115) (dominant negative deletion)
atggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagataa

SEQ ID NO: 20 - DNA, 679 bp, artificial sequence
  P25739 G1792 nucleic acid from A. thaliana with site-directed point mutation #2
gtcgagaatccatagatctcttattaaataacagtgctgaccaagctcttacaaagcaaaccaatctagaacaccaaagttaatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttgggcgataaggttcttgaagaacttcttgattcagaagaaaggaagagataatcacgattagttttgttttgatattttatgtggcactgttgtggctacctacgtgcattatgtgcatgtataggtcgcttgattagtactttataacatgcatgccacgaccataaattgtaagagaagacgtactttgcgttttcatgaaatatgaatgttagatggtttgagtac

SEQ ID NO: 21 - DNA, 679 bp, artificial sequence
  P25740, G1792 nucleic acid from A. thaliana with point mutation #3
gtcgagaatccatagatctcttattaaataacagtgctgaccaagctcttacaaagcaaaccaatctagaacaccaaagttaatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttggggaagaacttcttgattcagaagaaaggaagagataatcacgattagttttgttttgatattttatgtggcactgttgtggctacctacgtgcattatgtgcatgtataggtcgcttgattagtactttataacatgcatgccacgaccataaattgtaagagaagacgtactttgcgttttcatgaaatatgaatgttagatggtttgagtac

SEQ ID NO: 22 - DNA, 679 bp, artificial sequence
  P25741  G1792 nucleic acid from A. thaliana with site-directed point mutation #4 (G1792 is from A. thaliana)
gtcgagaatccatagatctcttattaaataacagtgctgaccaagctcttacaaagcaaaccaatctagaacaccaaagttaatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttggggattcagaagaaaggaagagataatcacgattagttttgttttgatattttatgtggcactgttgtggctacctacgtgcattatgtgcatgtataggtcgcttgattagtactttataacatgcatgccacgaccataaattgtaagagaagacgtactttgcgttttcatgaaatatgaatgttagatggtttgagtac

SEQ ID NO: 23 - DNA, 833 bp, artificial sequence
  P25083 nucleic acid construct comprising a 35S::G1792-GAL4 direct promoter fusion (G1792 is from A. thaliana)
taaggaagttcatttcatttggagaggacacgctgacaagctgactctagcagatctggtaccatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagagcggccgccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatatacctatctattcgatgatgaagataccccaccaaacccaaaaaaagagtag

SEQ ID NO: 24 - DNA, 909 bp, artificial sequence
  P25093 nucleic acid construct comprising a 35S::GAL4-G1792 direct promoter fusion (G1792 is from A. thaliana)
atgcccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagggtaccatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagataagcggccgctctagacaggcctcgtaccggatcctctagctagagctttcgttcgtatcatcggtttcgacaacgttcgtcaagttcaatgcatcagtttcattgcgcacacaccagaatcctcgagaattttgagata

SEQ ID NO: 25 - DNA, 1161 bp, artificial sequence
  P25271 nucleic acid construct, 35S::G1792- green fluorescent protein fusion directly fused to a 35S promoter
atggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagatgcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagggatcctctag

SEQ ID NO: 26 - DNA, 981 bp, artificial sequence
  P25678, G28 nucleic acid from A. thaliana with point mutation #1
gtcgacccacgcgtccggaaatctcaacaagaaccaaaccaaacaacaaaaaaacattcttaataattatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcgacgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattaaaaaaaaaaaaaaaaaa

SEQ ID NO: 27 - DNA, 981 bp, artificial sequence
  P25680, G28 nucleic acid from A. thaliana with point mutation #3
gtcgacccacgcgtccggaaatctcaacaagaaccaaaccaaacaacaaaaaaacattcttaataattatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgaggatgcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattaaaaaaaaaaaaaaaaaa

SEQ ID NO: 28 - DNA, 981 bp, artificial sequence
  P25682, G28 nucleic acid from A. thaliana with point mutation #5
gtcgacccacgcgtccggaaatctcaacaagaaccaaaccaaacaacaaaaaaacattcttaataattatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcgacgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgaggatgcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattaaaaaaaaaaaaaaaaaa

SEQ ID NO: 29 - DNA, 865 bp, artificial sequence
  P25684, G28 nucleic acid from A. thaliana with point mutation #7
taataaatatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcgacgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggagattttgaggatgcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttca

SEQ ID NO: 30 - DNA, 1161 bp, artificial sequence
  P21143 nucleic acid construct of 35S::G28-GAL4 C-terminal fusion(G28 from A. thaliana fused at its C-terminus to GAL4)
gttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttagaattccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagtaa

SEQ ID NO: 31 - DNA, 1250 bp, artificial sequence
  P21196  nucleic acid construct of 35S::GAL4- G28 direct promoter N-terminal fusion (G28 from A. thaliana fused at its N-terminus to GAL4)
atgcccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagggtacctcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattaaaaaaaaaaaaaaaaaa

SEQ ID NO: 32 - DNA, 548 bp, artificial sequence
  P21276 overexpression construct encoding truncated version of G867 protein (residues 139 to 309 of wild- type protein) containing the B3 domain but not the AP2 domain (dom neg deletion in secondary domain)
tcgacctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttgaccggagagttcgc

SEQ ID NO: 33 - DNA, 419 bp, artificial sequence
  P21275 overexpression construct encoding truncated version of G867 protein (residues 36 to 165 of wild- type protein) containing the AP2 domain (dom neg deletion)
tcgaccttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgtagttaacgtcggc

SEQ ID NO: 34 - DNA, 1389 bp, artificial sequence
  P21193 nucleic acid construct encoding a G867 C-terminal GAL fusion 35S::G867-GAL4 (overexpression construct encoding a G867 clone with a GAL4 transactivation domain fused at the C terminus)
taaatggaatcgagtagcgttgatgagagtactacaagtacaggttccatctgtgaaaccccggcgataactccggcgaaaaagtcgtcggtaggtaacttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttcaccggagagttcaagaaacgacgtcgtaggaaacaaaagagtgaacgatactgagatgttatcgttggtgtgtagcaagaagcaacgcatctttcacgcctcgcaattccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagtaa

SEQ ID NO: 35 - DNA, 1526 bp, artificial sequence
  P21201, nucleic acid construct encoding a G867 N-terminal GAL fusion 35S::GAL4-G867 (overexpression construct encoding a G867 clone that has a GAL4 transactivation domain fused at the N terminus)
atgcccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagggtaccgtcgacgaatcgagtagcgttgatgagagtactacaagtacaggttccatctgtgaaaccccggcgataactccggcgaaaaagtcgtcggtaggtaacttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttcaccggagagttcaagaaacgacgtcgtaggaaacaaaagagtgaacgatactgagatgttatcgttggtgtgtagcaagaagcaacgcatctttcacgcctcgtaacaactcttcttctttttttttcttttgttgttttaataatttttaaaaactccattttcgttttctttatttgcatcggtttctttcttcttgtttaccaaaggttcatgagttgtttttgttgtattgatgaactg

SEQ ID NO: 36 - DNA, 1776 bp, artificial sequence
  P25301 nucleic acid construct encoding a 35S:: G867-GFP direct fusion
atggaatcgagtagcgttgatgagagtactacaagtacaggttccatctgtgaaaccccggcgataactccggcgaaaaagtcgtcggtaggtaacttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttcaccggagagttcaagaaacgacgtcgtaggaaacaaaagagtgaacgatactgagatgttatcgttggtgtgtagcaagaagcaacgcatctttcacgcctcgtgcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagggatcctctag

SEQ ID NO: 37 - DNA, 505 bp, artificial sequence
  P25732 nucleic acid construct encoding G47 from A. thaliana with point mutation #1
cttcttcttcacatcgatcatcatacaacaacaaaaaatggattacagagaatccaccggtgaaagtcagtcaaagtacaaaggaatccgtcgtcggaaatggggcaaatgggtatcagagattagagagccgggaactcgtgaccgtctctggttaggttcattctcaacagcagaaggtgccgccgtagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgatttatctcgacggtcatgatcacgtttgatctt

SEQ ID NO: 38 - DNA, 505 bp, artificial sequence
  P25733 nucleic acid construct encoding G47 from A. thaliana with point mutation #2
cttcttcttcacatcgatcatcatacaacaacaaaaaatggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccagagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgatttatctcgacggtcatgatcacgtttgatctt

SEQ ID NO: 39 - DNA, 505 bp, artificial sequence
  P25735 nucleic acid construct encoding G47 from A. thaliana with point mutation #4
cttcttcttcacatcgatcatcatacaacaacaaaaaatggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccgtagcacacgacacagctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgatttatctcgacggtcatgatcacgtttgatctt

SEQ ID NO: 40 - DNA, 825 bp, artificial sequence
  P25186 overexpression construct encoding a G47 from A. thaliana clone that has a GAL4 transactivation domain fused at the N terminus(35S::GAL4-G47 N-terminal GAL fusion)
atgcccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagggtaccgtcgacatggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccgtagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgatttatctcgacggtcatgatcacgtttgatctt

SEQ ID NO: 41 - DNA, 1176 bp, artificial sequence
  P25279 nucleic acid construct encoding a 35S::G47-GFP fusion directly fused to the 35S promoter, and a KanR marker
atggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccgtagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttgcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagggatcctctag

SEQ ID NO: 42 - DNA, 1281 bp, artificial sequence; identical to SEQ ID NO: 7
  P7140 nucleic acid construct comprising opLexA::G867(G867 is from A. thaliana)

SEQ ID NO: 43 - DNA, 724 bp, artificial sequence; identical to SEQ ID NO: 1
  P3366 nucleic acid construct comprising (opLexA)::G912 (G912 is from A. thaliana)

SEQ ID NO: 44 - DNA, 4276 bp, artificial sequence
  P6506 nucleic acid construct comprising 35S:: LexA-GAL4 (LexA binding protein-GAL4 transcription activator)
catgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcggcggccgcaagacccttcctctatataaggaagttcatttcatttggagaggacacgctcgagtataagagctcatttttacaacaattaccaacaacaacaaacaacaaacaacattacaattacatttacaattaccatggaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccacttctggcgcaacagcatattgaaggtcattatcaggtcgatccttccttattcaagccgaatgctgatttcctgctgcgcgtcagcgggatgtcgatgaaagatatcggcattatggatggtgacttgctggcagtgcataaaactcaggatgtacgtaacggtcaggtcgttgtcgcacgtattgatgacgaagttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatctagaacatatccatatctaatcttacctcgactgctgtatataaaaccagtggttatatgtccagtactgctgtatataaaaccagtggttatatgtacagtacgtcgatcgatcgacgactgctgtatataaaaccagtggttatatgtacagtactgctgtatataaaaccagtggttatatgtacagtacgtcgaggggatgatcaagacccttcctctatataaggaagttcatttcatttggagaggacacgctcgagtataagagctcatttttacaacaattaccaacaacaacaaacaacaaacaacattacaattacatttacaattaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgca